(19) United States
(12) Patent Application Publication
(10) Pub. No.: US 2003/0087292 A1
(43) Pub. Date: May 8, 2003

(54) Methods and systems for promoting interactions between probes and target molecules in fluid in microarrays

(76) Inventors: Shiping Chen, Fremont, CA (US); Jianming Xiao, Fremont, CA (US); Kaijun Li, San Jose, CA (US); Hoang M. Nguyen, San Jose, CA (US)

Correspondence Address: Charles D. Holland, Morrison & Foerster LLP, 755 Page Mill Road, Palo Alto, CA, 94304-1018, US

(21) Appl. No.: 10/264,892

(22) Filed: Oct. 4, 2002

Related U.S. Application Data
(60) Provisional application No. 60/327,686, filed on Oct. 4, 2001.
(60) Provisional application No. 60/402,371, filed on Aug. 8, 2002.
(60) Provisional application No. 60/357,392, filed on Feb. 15, 2002.

(51) Int. Cl. C12Q 1/68; C12M 1/34

(52) U.S. Cl. 435/6; 435/287.2; 435/286.5

(57) ABSTRACT

Methods and apparatus for promoting interactions between an array of probes deposited on a microarray substrate and target molecules in a target liquid are provided. A microarray apparatus can include a substrate having an array of probes deposited on a surface of the substrate for interaction with a target molecule in a target liquid. The apparatus also includes a cover coupled to the substrate to form a reaction chamber therebetween, wherein the array of probes is contained within the reaction chamber and the substrate and the cover are movable relative to each other.

### CROSS-REFERENCES TO RELATED APPLICATIONS

This application claims the benefit of priority to U.S. patent applications Ser. No. 60/327,686, entitled “Methods and Apparatus for Microarray Hybridization” by Shiping Chen et al., filed Oct. 4, 2001, and Ser. No. 60/402,371, entitled “Micro-Channels for Hybridization Enhancement” by Shiping Chen, filed Aug. 8, 2002. The above applications are incorporated by reference herein in their entireties as if fully set forth below for all purposes.

### FIELD OF THE INVENTION

The invention relates generally to the field of biochemical analysis in which it is desirable to facilitate interaction between immobilized probes with target molecules in a fluid.

### BACKGROUND OF THE INVENTION

Many applications in bio-chemical study involve the binding of target molecules in a target liquid to probes that are immobilized on a substrate surface. The immobilized probes can be, for example, oligonucleotides, peptides, polypeptides, proteins, antibodies, or other molecules capable of reacting with the target molecules.

FIG. 1 shows one widely used apparatus for microarray hybridization experiments. A cover slip having small risers provided on each edge is placed on a microscope substrate slide, on which the microarray probes are deposited. The target sample is introduced into the space between the cover slip and the substrate slide, and this assembly is then sealed in a small chamber, which is then placed in a water bath and maintained at a constant temperature for several hours.

An advantage of such a hybridization device is its low cost and simplicity. However, it also has several disadvantages. First, the sensitivity of the system may be limited. The narrow space between the cover slip and the substrate (typically 20 μm to 50 μm in height) restricts the flow of sample fluid and limits the mobility of target molecules. For any individual probe in the microarray, only complementary target molecules that are within a small area centered around the probe spot are likely to hybridize with the probe. As shown in FIG. 2, the actual effective sample volume for any probe can be expressed as v=πr²h, where r is the radius of the above mentioned area centered around the probe spot and h is the height of fluid space between the cover slip and the substrate. Such a volumetric restriction can significantly reduce the sensitivity of the detection. Assuming a typical r of 200 μm and the entire cover slip area of 20 mm×20 mm, the effective volume is only 0.03% of the total volume. This means that the detection sensitivity is reduced by a factor of 3000.

Another possible disadvantage is that there can be variation of hybridization sensitivity between chips. The amount of target molecules available for hybridization is proportional to the volume of sample fluid in the effective space described above and the effective sample volume is in turn proportional to the height of the gap between the slip and substrate. Because it is very difficult to precisely control the gap height, the chip-to-chip hybridization consistency can be low with this method.

In addition, the hybridization process can be slow. Because the sample fluid is quiescent, the target molecules rely on random Brownian motion to meet and hybridize with complimentary probes. This can result in a very long hybridization process (usually overnight).

### SUMMARY OF THE INVENTION

In accordance with embodiments of the present invention, a microarray apparatus is provided. The apparatus comprises a substrate having an array of probes deposited on a surface of the substrate for interaction with a target molecule in a target liquid; and a cover coupled to the substrate to form a reaction chamber therebetween, wherein the array of probes is contained within the reaction chamber and the substrate and the cover are movable relative to each other.

In accordance with further embodiments of the present invention, a microarray apparatus is provided. The apparatus comprises a reaction chamber having an interior cavity and an array of probes deposited on an inner surface of the interior cavity for reaction with a target molecule in a target liquid; a magnetically reactive mixing member contained in the reaction chamber; and a magnetic field generator for moving the magnetically reactive mixing member through the target liquid.

In accordance with further embodiments of the present invention, a microarray apparatus is provided. The apparatus comprises a reaction chamber having an interior cavity; a target liquid contained within the interior cavity of the reaction chamber; a volume exclusion liquid contained within the interior cavity; and an array of probes deposited on an inner surface of the interior cavity of the reaction chamber for reaction with a target molecule in the target liquid.

In accordance with further embodiments of the present invention, a microarray apparatus is provided. The apparatus comprises a reaction chamber having an interior cavity; an array of probes deposited on an inner surface of the interior cavity for reaction with a target molecule in a target liquid; and a transducer for directing acoustic waves into the interior cavity of the reaction chamber.

In accordance with further embodiments of the present invention, a microarray apparatus is provided. The apparatus comprises a reaction chamber having an interior cavity; an array of probes deposited on an inner surface of the interior cavity for reaction with a charged target molecule in a target liquid; and a voltage generator for generating a voltage across the interior cavity to move the charged target molecule.

In accordance with further embodiments of the present invention, a microarray apparatus is provided. The apparatus comprises a reaction chamber having an interior cavity; an array of probes deposited on an inner surface of the interior cavity for reaction with a charged target molecule in a target liquid; and a temperature control mechanism for generating a temperature gradient across the interior cavity of the reaction chamber.

In accordance with further embodiments of the present invention, a microarray apparatus is provided. The apparatus comprises a substrate; an array of probes deposited on a surface of the substrate; and a cover having a channel with a width smaller than a width of the array of probes, said cover being coupled to the substrate such that said channel and said substrate define a channel cavity such that a target fluid flowing through the channel cavity contacts each probe in the array of probes.

In accordance with further embodiments of the present invention, a microarray apparatus is provided. The apparatus comprises a reaction chamber having an interior cavity; an array of probes deposited on an inner surface of the interior cavity of the reaction chamber for reaction with a target molecule in a target liquid; and a shape modulator for varying the shape of the interior cavity.

In accordance with further embodiments of the present invention, a microarray apparatus is provided. The apparatus comprises a chamber filled with a combination of a volume exclusion liquid and a target liquid.

In accordance with further embodiments of the present invention, a microarray apparatus is provided. The apparatus comprises a substrate having a plurality of arrays of probes deposited on a surface the substrate; and a cover coupled with the substrate such that the cover and the substrate form a chamber over each array of probes, said cover having an inlet for introducing a target liquid into the chamber.

In accordance with further embodiments of the present invention, a method for promoting interaction between a target molecule in a target liquid and an array of probes deposited on a surface of a substrate is provided. The method comprises loading the target liquid on top of the array of probes; positioning a cover on top of the target liquid; and creating a relative motion between the substrate and the cover for generating movement of the target molecule.

In accordance with further embodiments of the present invention, a method for promoting interaction between a target molecule in a target liquid and an array of probes deposited on an interior surface of a reaction chamber for confining the target liquid is provided. The method comprises loading the target liquid in the reaction chamber; and applying a magnetic force to move a magnetically reactive mixing member contained within the reaction chamber to generate motion of the target molecule.

In accordance with further embodiments of the present invention, a method for promoting interaction between a target molecule in a target liquid and an array of probes deposited on an interior surface of a reaction chamber for confining the target liquid is provided. The method comprises loading the target liquid into the reaction chamber; loading a volume exclusion liquid into the reaction chamber; and agitating the reaction chamber to cause relative movement between the volume exclusion liquid and the target liquid.

In accordance with further embodiments of the present invention, a method for promoting interaction between a target molecule in a target liquid and an array of probes deposited on an interior surface of a reaction chamber is provided. The method comprises loading the target liquid into the reaction chamber; and directing acoustic waves through the target liquid to generate motion of the target molecule.

In accordance with further embodiments of the present invention, a method for promoting interaction between a charged target molecule in a target liquid and an array of probes deposited on a surface of a substrate is provided. The method comprises loading the target liquid into the reaction chamber; and generating a voltage across the reaction chamber to generate motion of the charged target molecule contained within the target liquid.

In accordance with further embodiments of the present invention, a method for promoting interaction between a charged target molecule in a target liquid and an array of probes deposited on a surface of a substrate is provided. The method comprises loading the target liquid into the reaction chamber; and generating an electric field across the reaction chamber to generate motion of the charged target molecule contained within the target liquid.

In accordance with further embodiments of the present invention, a method for promoting interaction between a target molecule in a target liquid and an array of probes deposited on an interior surface of a reaction chamber for confining the target liquid is provided. The method comprises loading the target liquid in the reaction chamber; and generating a temperature gradient in the target fluid across the reaction chamber.

In accordance with further embodiments of the present invention, a method for promoting interaction between a target molecule in a target liquid and an array of probes deposited on a surface of a substrate is provided. The method comprises loading a target liquid into a channel, said channel having a width smaller than a width of the array of probes; and passing the target liquid through the channel across all of the probes in the probe array.

In accordance with further embodiments of the present invention, a method for promoting interaction between a target molecule in a target liquid and an array of probes deposited on an interior surface of a reaction chamber for confining the target liquid is provided. The method comprises loading the target liquid into an interior cavity of the reaction chamber; and changing the shape of the interior cavity of the reaction chamber to generate a pressure wave in the target liquid.

In accordance with further embodiments of the present invention, a microarray apparatus is provided. The apparatus comprises a reaction chamber comprising a substrate having an array of probes deposited thereon, and a cover coupled to the substrate to form an interior cavity of the reaction chamber between the substrate and the cover; an array of probes deposited on an inner surface of the interior cavity for reaction with a charged target molecule in a target liquid; and a flow inducing mechanism for inducing flow of the target liquid without physically translating either the substrate or the cover.

In accordance with further embodiments of the present invention, a method for promoting interaction between an array of probes deposited on a surface of a substrate and a target molecule in a target liquid contained within a reaction chamber formed by the substrate and a cover is provided. The method comprises loading the target liquid in the reaction chamber; and inducing movement of the target molecules in the target liquid without physically translating either the substrate or the cover.

Other features and aspects of the invention will become apparent from the following detailed description, taken in conjunction with the accompanying drawings which illustrate, by way of example, the features in accordance with embodiments of the invention. The summary is not intended to limit the scope of the invention, which is defined solely by the claims attached hereto.

### BRIEF DESCRIPTION OF THE DRAWINGS

FIG. 1 illustrates a prior art microarray hybridization device.

FIG. 2 illustrates the effective sample volume in existing microarray hybridization devices.

FIG. 3 is a perspective view of a capillary bundle in accordance with embodiments of the present invention.

FIG. 4 illustrates a compound loading station in which a pressure chamber containing a compound library in microtiter plates is coupled to capillary bundles.

FIG. 5 illustrates another parallel fluid delivery method utilizing gravity as the driving force.

FIG. 6A illustrates a method of functionalizing a substrate using protected-aldehyde silanization agent. FIG. 6B illustrates a method of functionalizing a substrate using maleimide silanization agent. FIG. 6C illustrates a method of activating the protected functional groups using light activation.

FIG. 7 illustrates a process for fabrication using a negative mask.

FIG. 8 illustrates a typical use of a chambered slide.

FIG. 9 illustrates a magnetic cover slip.

FIG. 10 illustrates a floating cover slip.

FIG. 11 illustrates use of a vibrating cover slip.

FIG. 12 illustrates use of a slide holder to immobilize the substrate slide while allowing the cover slip to move laterally in a relative larger area.

FIG. 13 illustrates an apparatus that moves the substrate slide to enhance movement of target molecules.

FIG. 14 illustrates a configuration of a sandwich hybridization chamber.

FIG. 15 illustrates use of a slide holder to maintain pressure in the slide stack.

FIG. 16 illustrates a configuration of the middle slide.

FIG. 17 illustrates a middle slide and cover slip as an integrated piece.

FIG. 18 illustrates a different configuration of the cover slip.

FIG. 19 illustrates use of a hybridization device with an integrated upper slide.

FIG. 20 illustrates a configuration using an immiscible fluid to prevent evaporation.

FIG. 21 illustrates forced circulation using a volume exclusion fluid in combination with gravitational or centrifugal force.

FIG. 22 illustrates use of magnetic beads to generate effective movement of target molecules.

FIG. 23 illustrates another use of magnetic beads to enhance hybridization.

FIG. 24 illustrates forced circulation using a magnetic fluid as the volume exclusion fluid and a magnetic field as the driving force.

FIG. 25 illustrates use of ultrasonic waves to generate effective movement of target molecules within the hybridization chamber.

FIG. 26 illustrates using an electric field to drive charged target molecules to migrate through the hybridization chamber along a predetermined route.

FIG. 27 illustrates an example voltage distribution and sequence that transports the target molecule along the electrode pads.

FIG. 28 illustrates another voltage sequence that transports the target molecules along the electrode pads.

FIG. 29 illustrates an apparatus with a simplified electrode configuration that makes use of an electrophoresis mechanism to drive target molecules to migrate across the hybridization chamber.

FIG. 30 illustrates use of upper electrode pads on the inner surface of the cover slip to reduce the voltage required for lateral transportation.

FIG. 31 illustrates coating a conductive layer near the upper surface of the substrate slide to help reduce the voltage required for vertical transportation of target molecules.

FIG. 32 illustrates use of upper electrode pads to transport target molecules towards the probes.

FIG. 33 illustrates use of an electric field gradient to drive the negatively charged molecules in the target fluid.

FIG. 34 illustrates use of Lorentz forces to move charged molecules in the target fluid.

FIG. 35 illustrates alternative electrode designs for using Lorentz forces to move charged molecules in the target fluid.

FIG. 36 illustrates use of localized heating/cooling to enhance movement of target molecules.

FIG. 37 illustrates pumping target fluid through microfluidic channels fabricated on the cover slip.

FIG. 38 illustrates a parallel channel design of microfluidic channels.

FIG. 39 illustrates use of external pressure chambers to force the target fluid to flow back and forth through the microfluidic channels.

FIG. 40 illustrates a micro-channel structure.

FIG. 41 illustrates surface treatment schemes for the micro-channel structure.

FIGS. 42a-42d illustrates micro-channel layout designs. FIG. 42a illustraes a single channel with a zip-zag route. FIG. 42b illustrates multiple parallel channels. FIG. 42c illustrates a interconnected two-dimensional channel matrix. FIG. 42d illustrates a surface pattern to generate random flow.

FIG. 43 illustrates a fluid reservoir where a capillary is used for metering of target fluid volume.

FIGS. 44a and 44b illustrate images of probe spots and the effect of the micro-channel structure on the areas available for hybridization.

FIG. 45 illustrates use of multiple pins on top of an elastic cover slip to generate effective movement of target molecules.

FIG. 46 illustrates a group of vibrating pins inserted into the target fluid to generate movement of target molecules.

FIGS. 47A and 47B illustrate a hybridization chamber for turbulent flow and volume exclusion hybridization. FIG. 47B illustrates the inner view of the chamber in FIG. 47A.

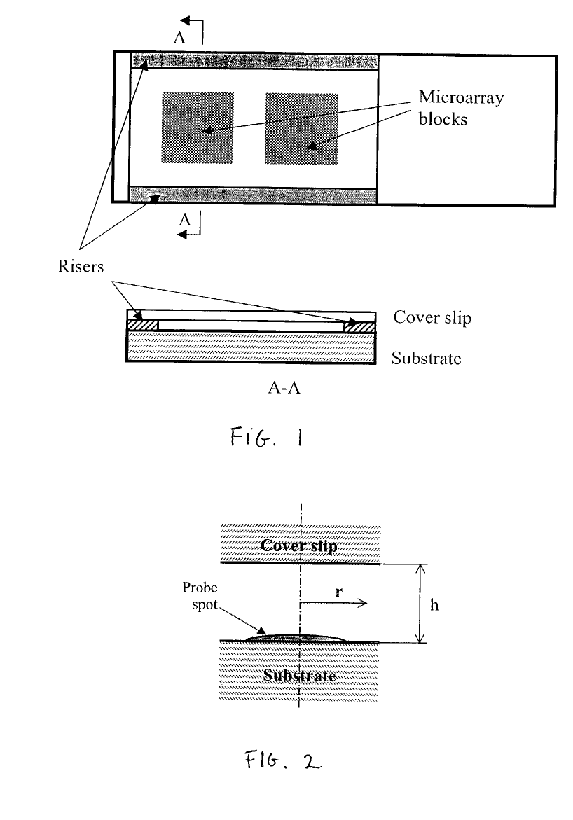

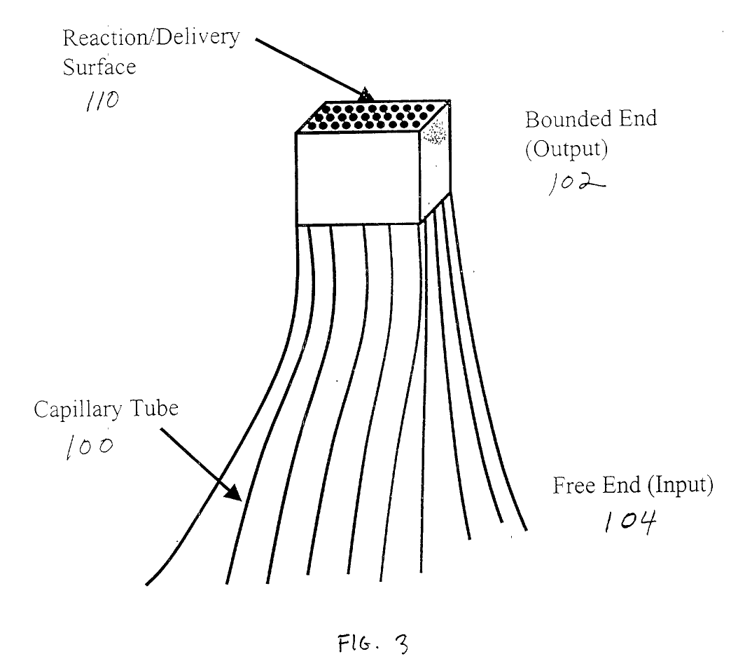

### DETAILED DESCRIPTION OF THE INVENTION

In accordance with embodiments of the present invention, systems and methods are provided which can facilitate interaction between probes immobilized on a substrate with target molecules in a fluid.

I. Probe Deposition

Probes can be immobilized on the surface of the microarray substrate by any method known in the art. For example, probes can be printed onto the surface using the capillary bundle system described herein below or the printing systems described in the following co-pending patent applications: U.S. application Ser. No. 10/080,274 entitled “Method and Apparatus Based on Bundled Capillaries for High Throughput Screening” by Shiping Chen et al., filed Feb. 19, 2002, which is a continuation-in-part of U.S. application Ser. No. 09/791,410 entitled “Method and Apparatus Based on Bundled Capillaries For High Throughput Screening” by Jianming Xiao et al., filed Feb. 22, 2001; U.S. application Ser. No. 09/791,994 entitled “Microarray Fabrication Techniques and Apparatus” by Shiping Chen et al., filed Feb. 22, 2001; U.S. application Ser. No. 09/791,998, entitled “Microarray Fabrication Techniques and Apparatus” by Shiping Chen et al., filed Feb. 22, 2001; U.S. Patent Application Publication 2002/0051979 A1 entitled “Microarray Fabrication Techniques and Apparatus” by Shiping Chen et al., filed Feb. 22, 2001; and PCT applications WO 01/62377 and WO 01/62378, which are incorporated by reference herein in their entirety as if fully set forth herein.

Printing systems described in these applications have a print head composed of one or more bundles of randomly bundled or discretely bundled capillaries. Each of the capillaries has a channel extending from the proximal end to the distal end of the capillary and has a channel-facing wall. This bundle of capillaries has a portion where at least the proximal ends of the capillaries are immobilized in a planar matrix and a facet is formed for printing. The immobilized portion can be sufficiently rigid that it may be used to print a probe or a group of probes upon a surface with minimal or no deformation (deformation may result in portions of the probes not being printed to the surface). The immobilized portion is therefore sufficiently rigid to ensure good contact with the surface across the portion of the facet in contact with the surface. The distal ends of the capillaries may be free or may be attached to reservoirs containing probes. The capillaries include, but are not limited to, fiber optic or other light-conducting capillaries, through which light as well as liquid can be conveyed, and other flexible or rigid capillaries. Probes can also be attached to the surface using, for example, covalent bonds in accordance with various methods known in the art.

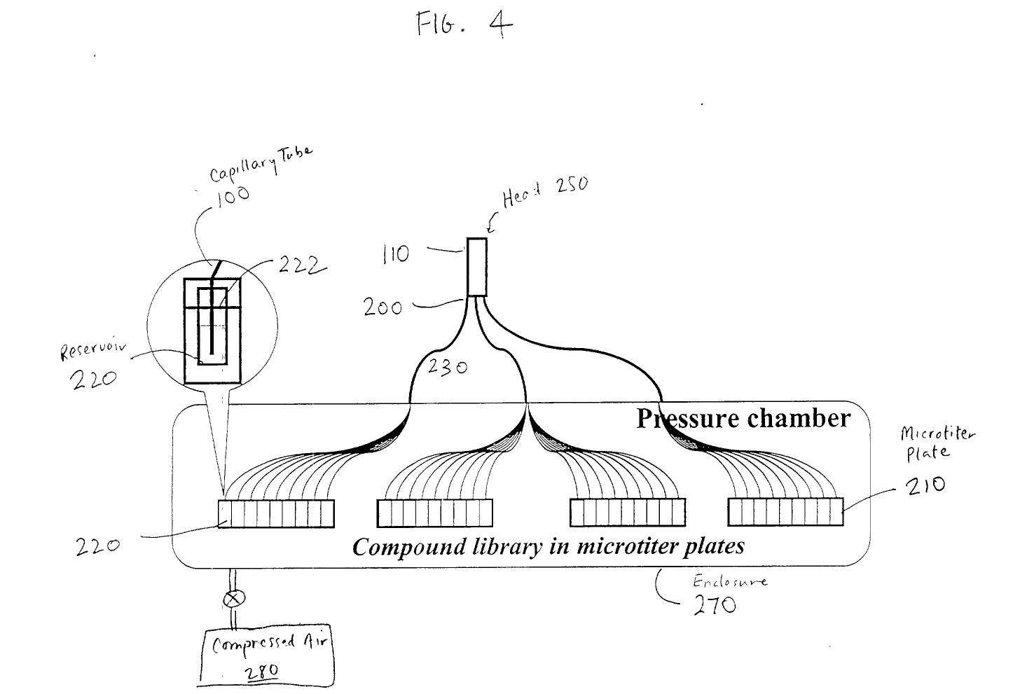

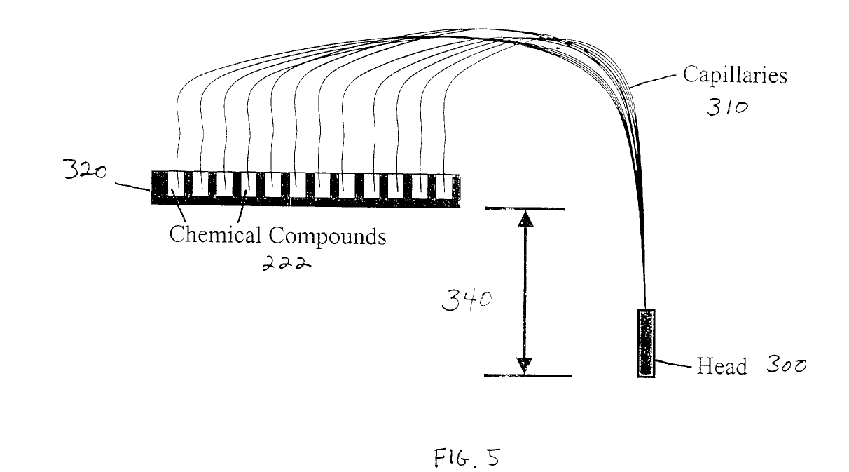

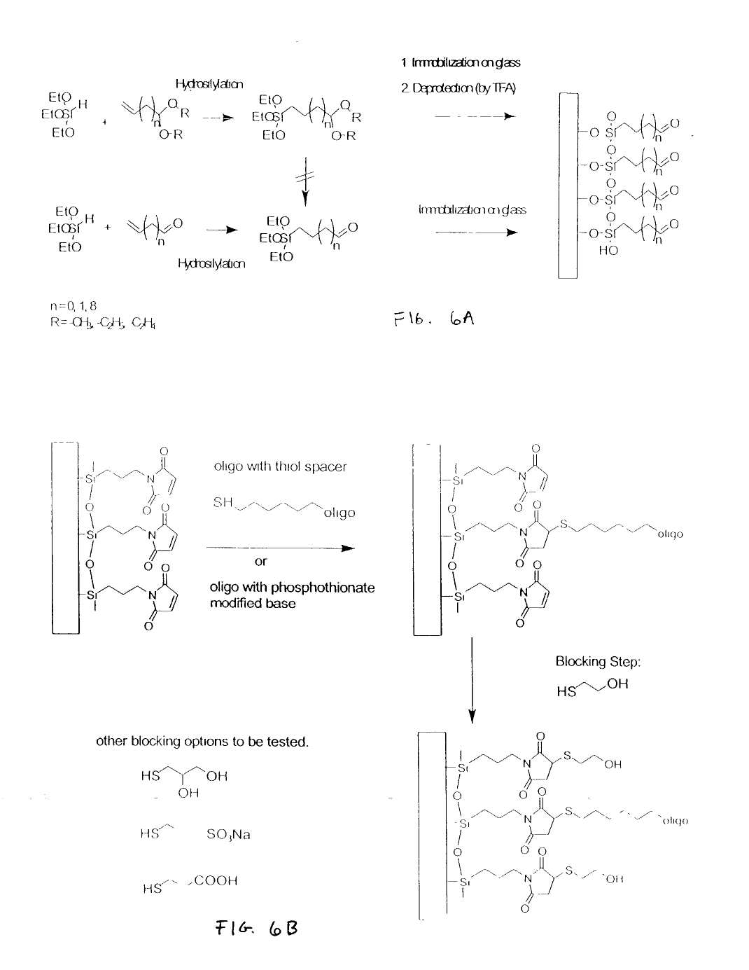

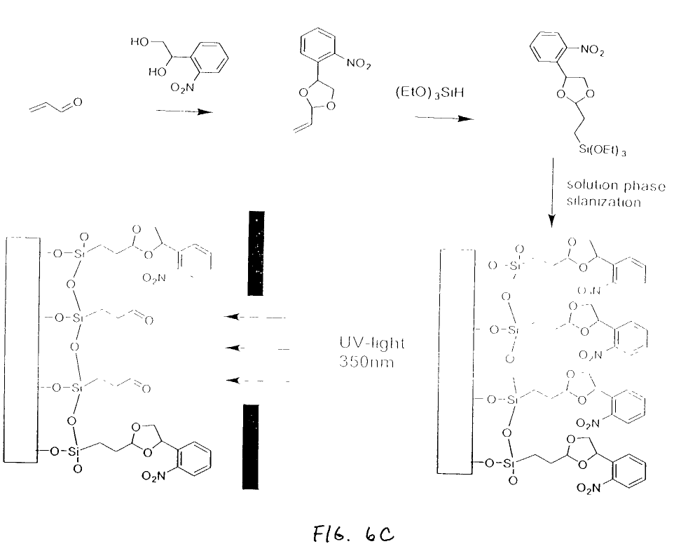

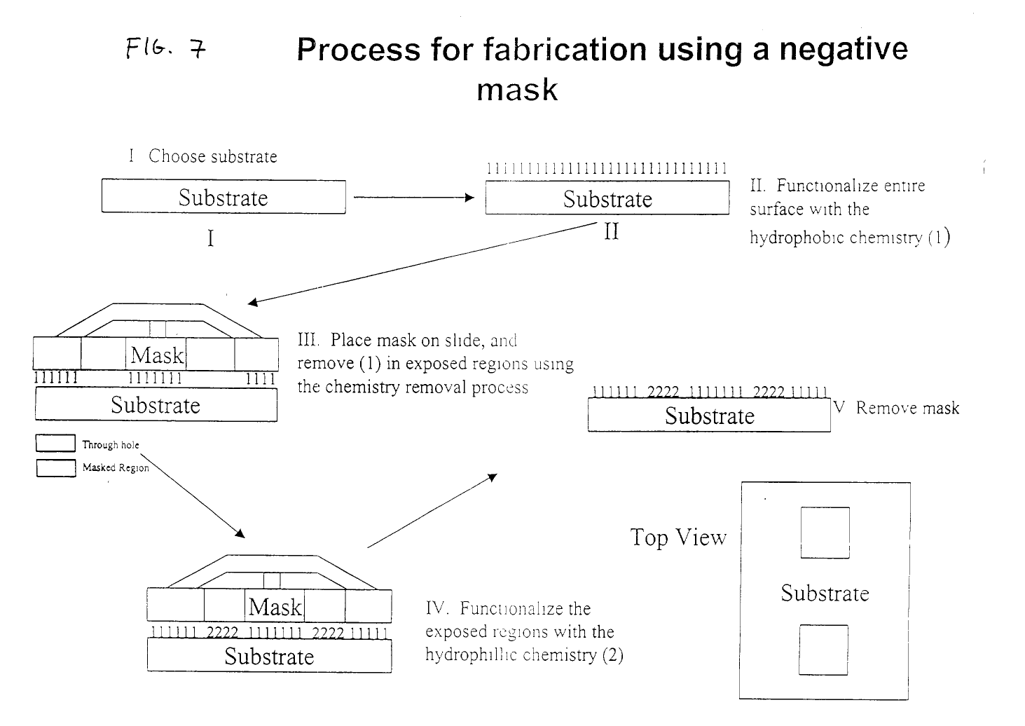

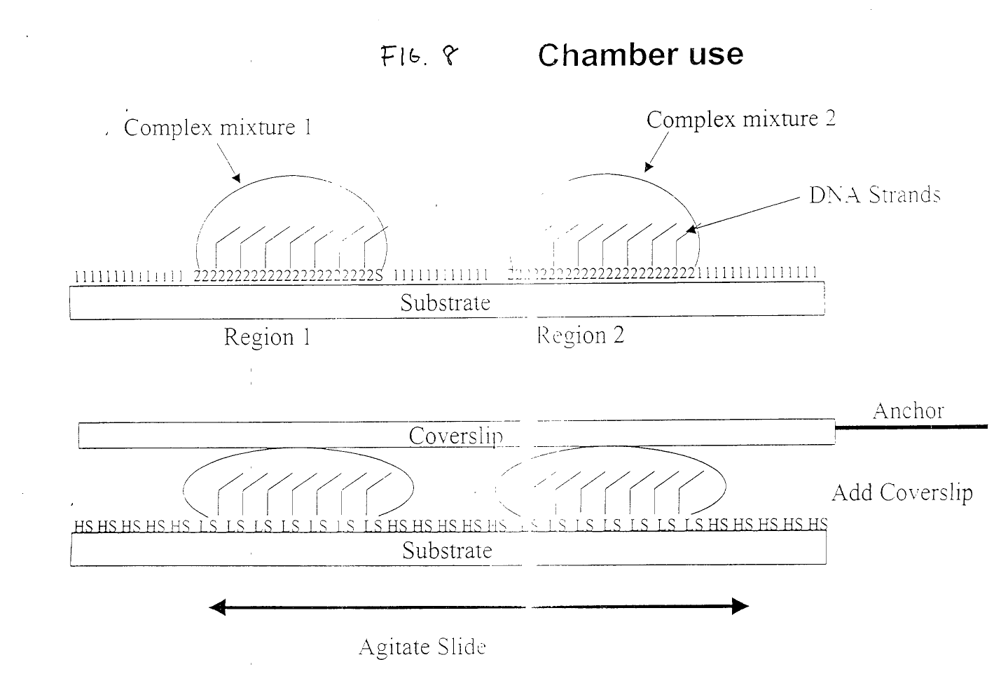

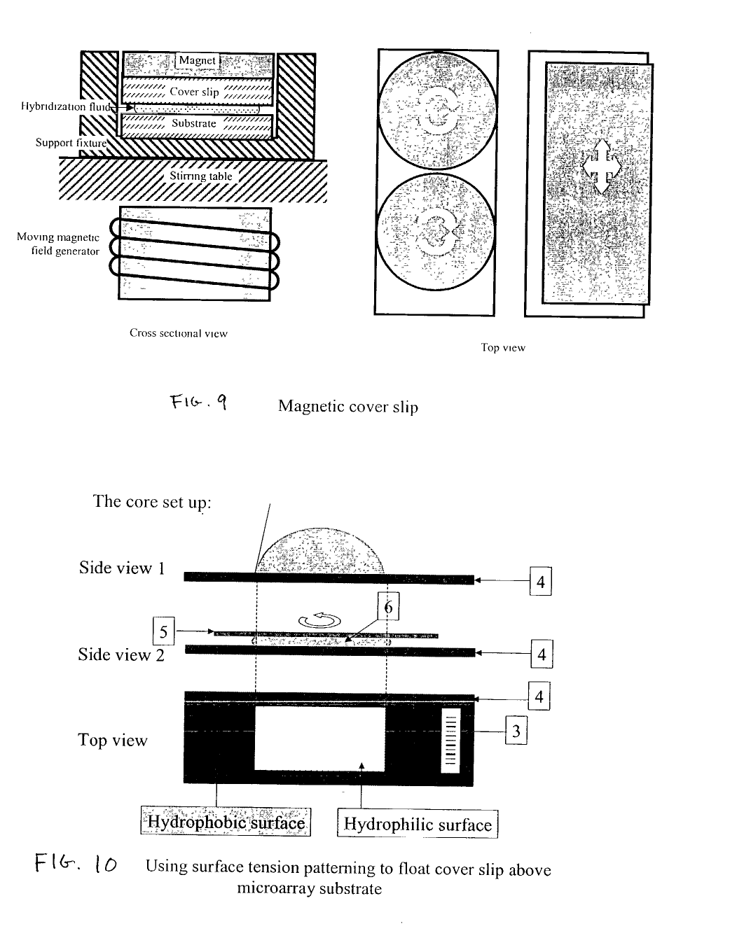

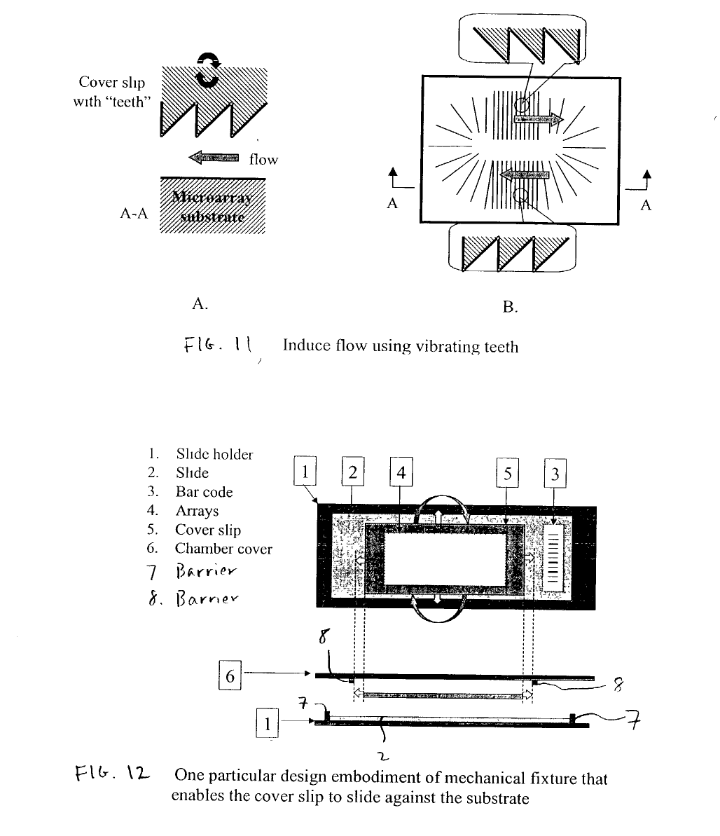

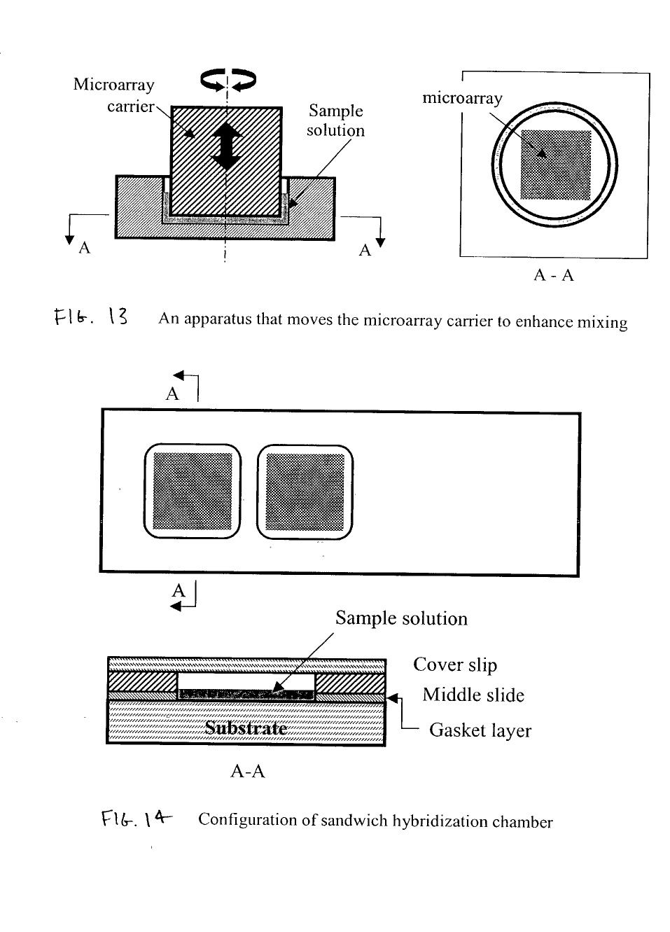

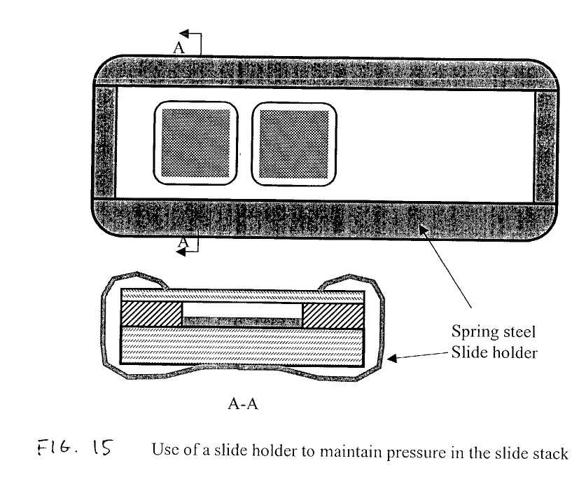

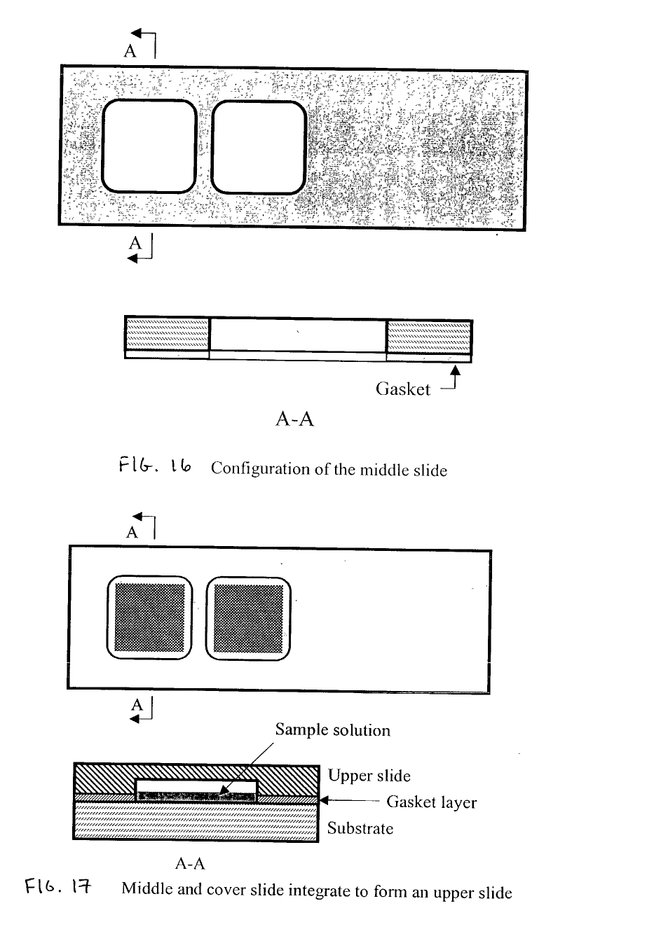

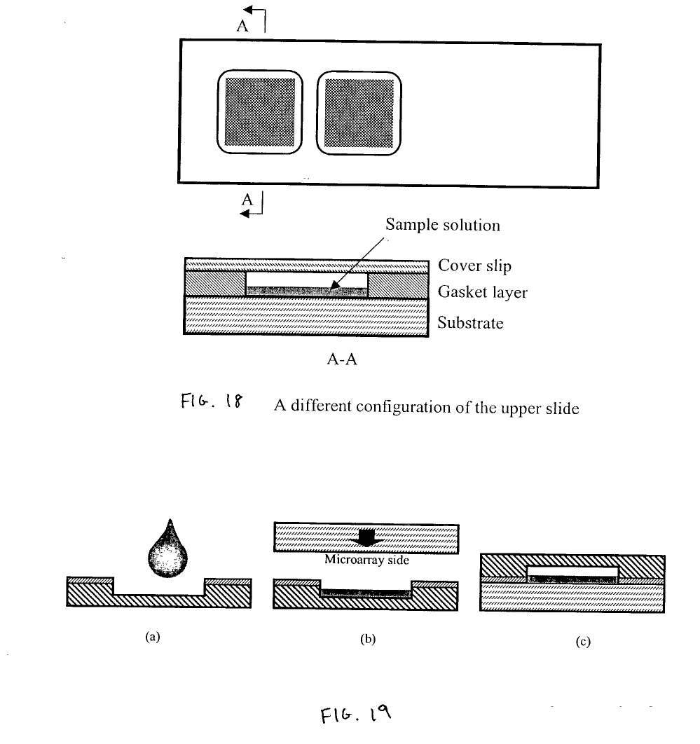

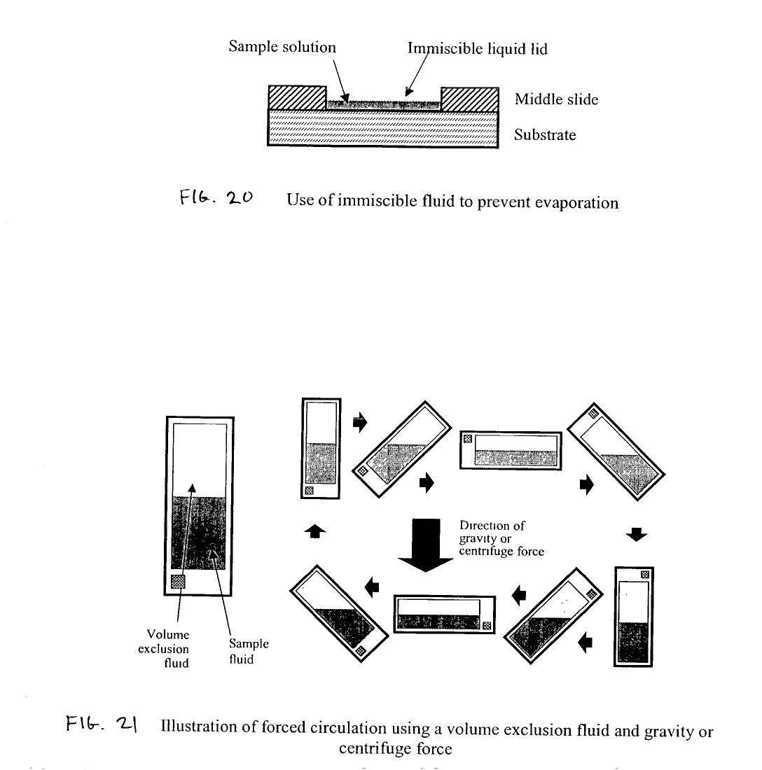

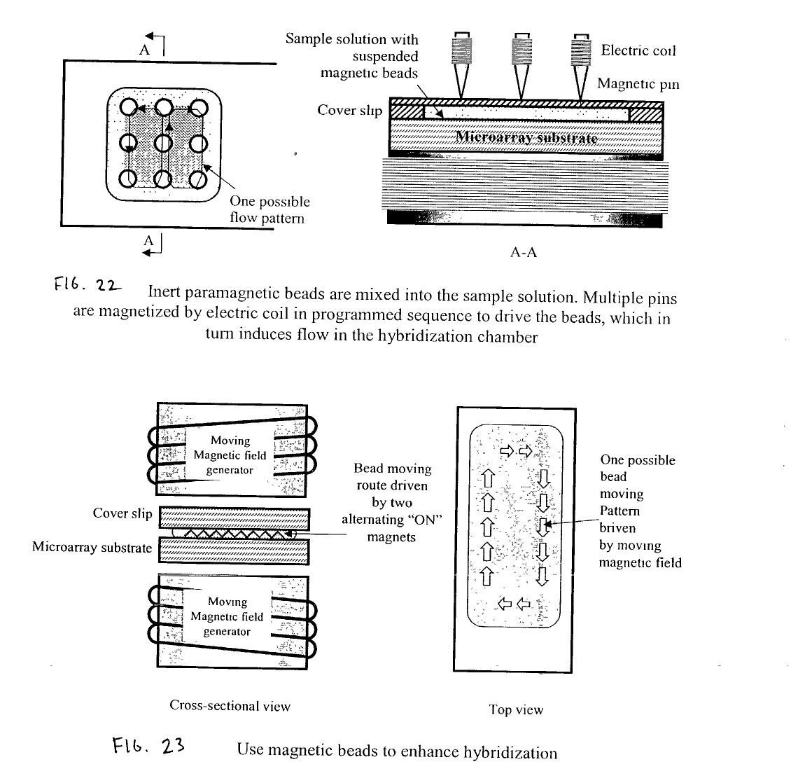

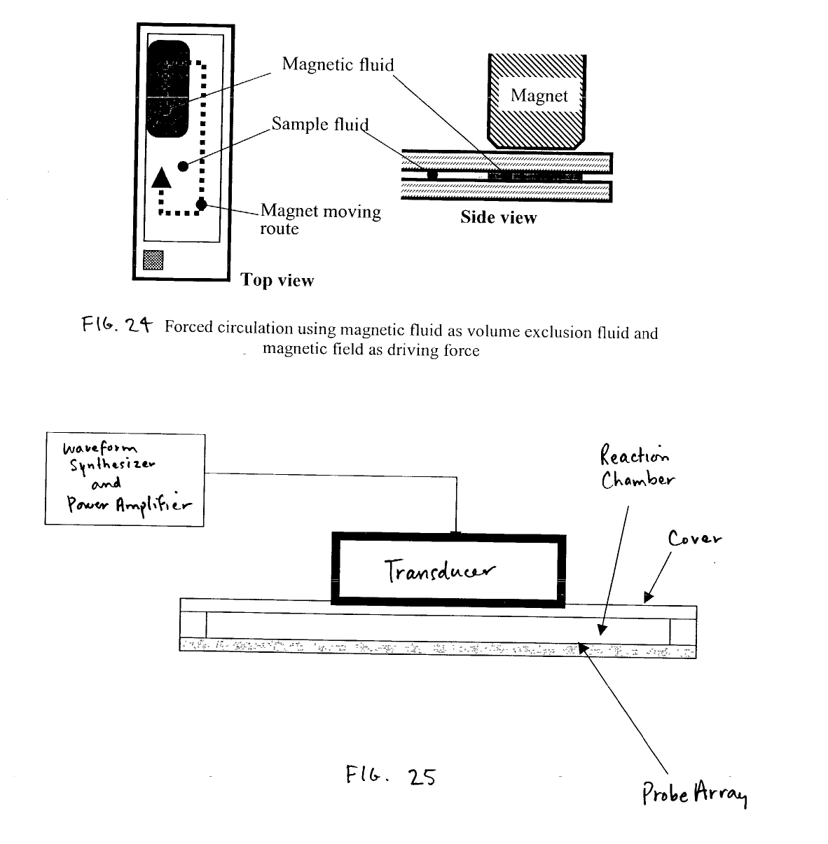

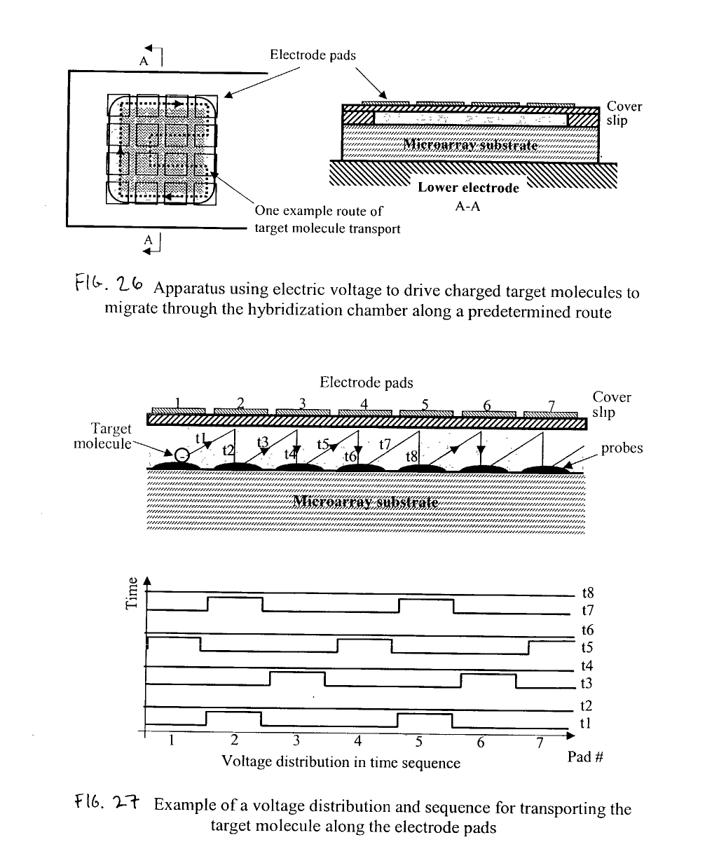

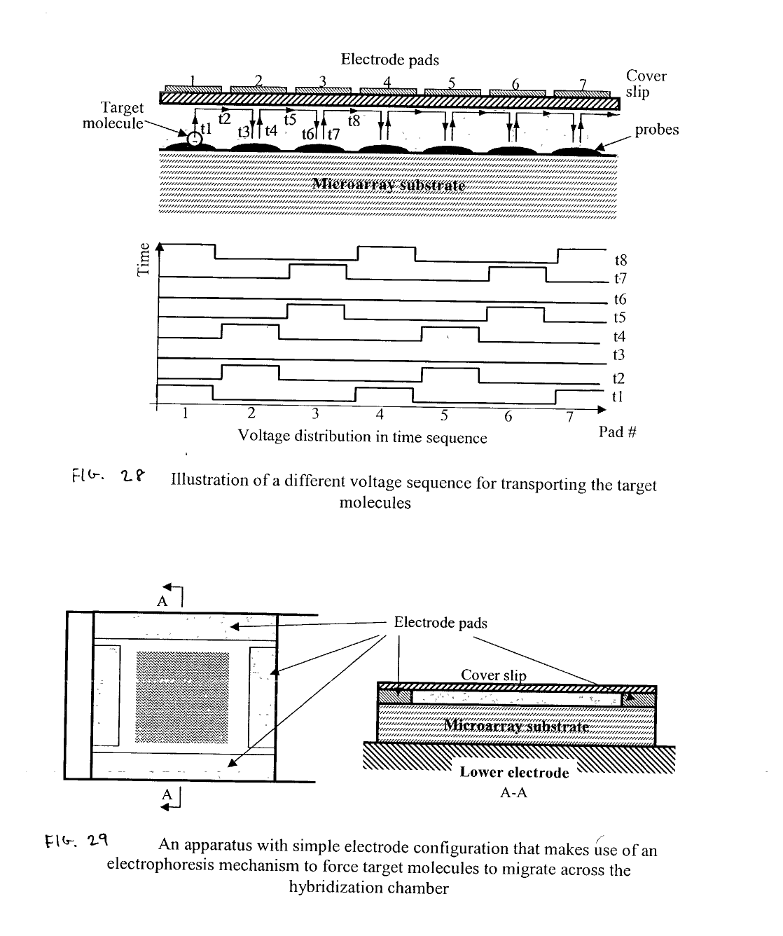

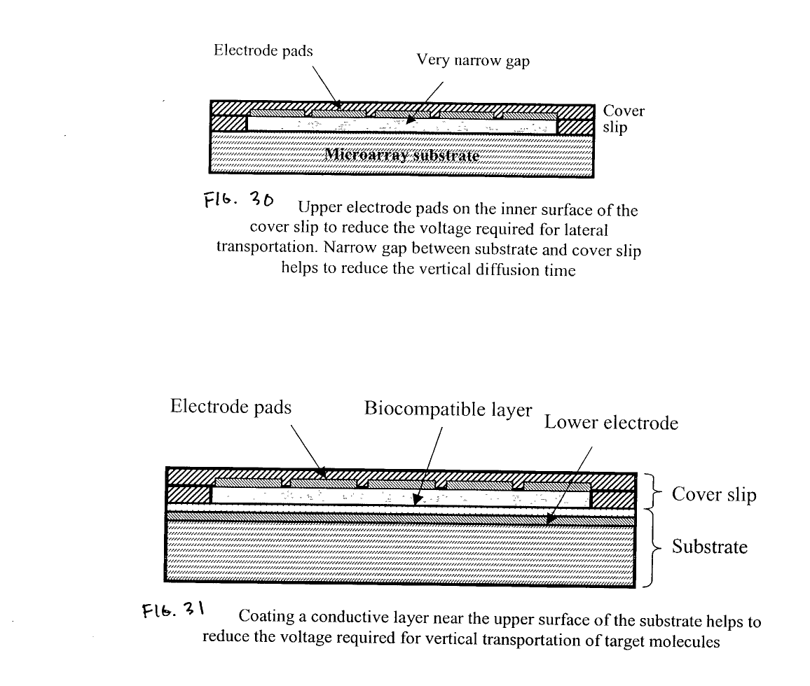

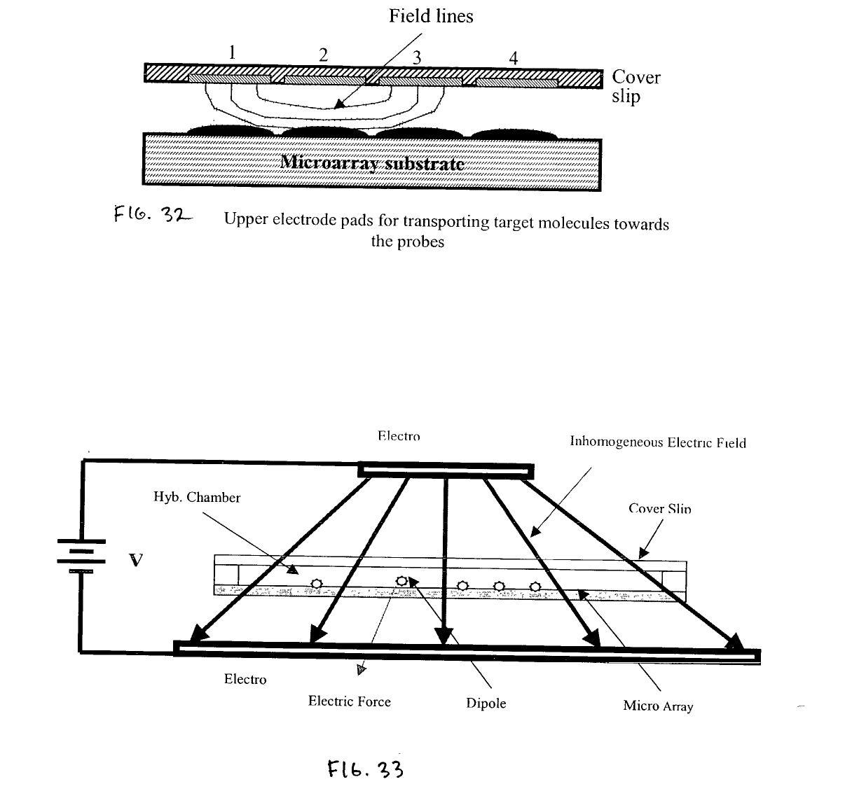

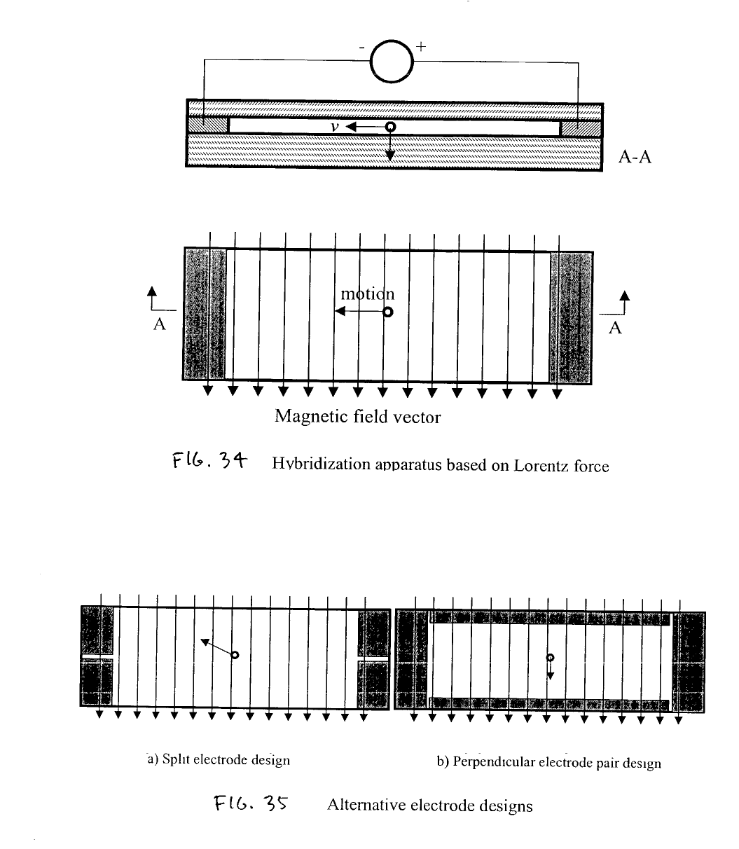

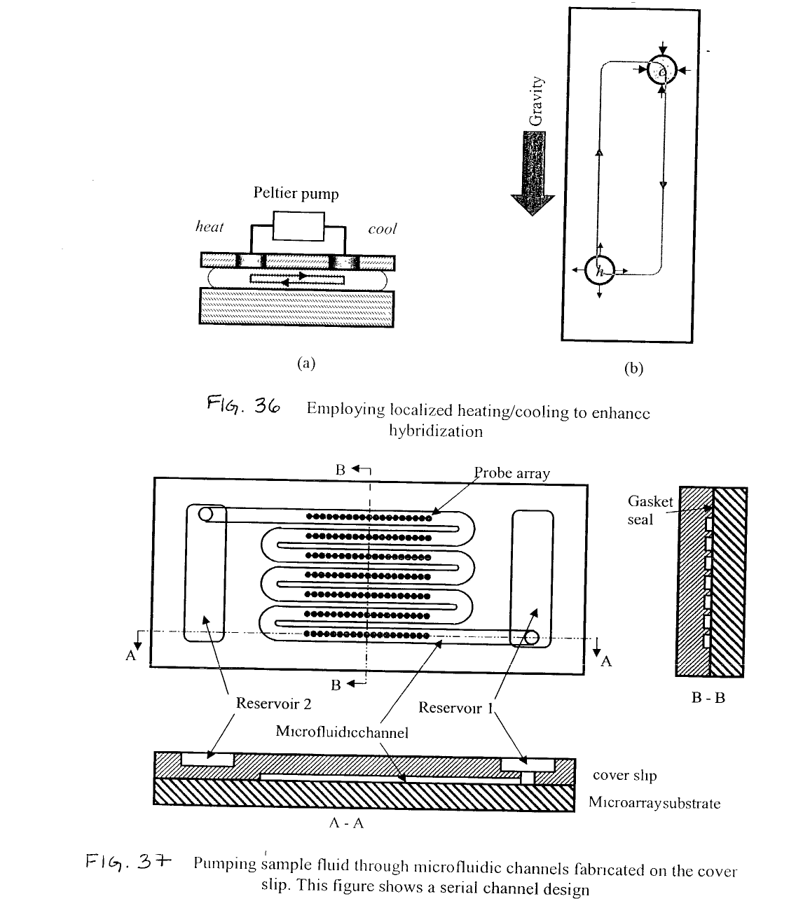

A capillary bundle 110 as depicted in FIG. 3 can be fabricated by using capillary tubes, such as those used for capillary electrophoresis. The tubes are bound at one end 102 to form a delivery head 110. The tubes may be gathered in either a random or an ordered fashion and bound, as discussed in the patent applications discussed above. The minimum number of tubes may depend upon the number of probes to be deposited. The number can be more than 100, preferably more than 10³, more preferably more than 10⁴, more preferably more than 10⁵ or more than 10⁶ or more than 10⁷).

The outer diameter of the capillary tubes can range from, for example, 5 to 500 micrometers, or preferably 30 to 300 micrometers, or more preferably 40 to 200 micrometers. The inner diameter of the tubes can range from, for example, 1 to 400 micrometers, or preferably 5 to 200 micrometers, or more preferably 10 to 100 micrometers. A capillary bundle as described herein may be attached or secured to a frame that is adapted to hold the capillary bundle in a print system. A delivery head may alternatively have a frame that holds a plurality of capillary bundles.

The capillary bundle has an input end 104 and an output end 102. Capillaries on the input end 104 may be left unbound and placed in contact with reservoirs, such as the wells in a microtiter plate, that hold the probes to be assayed such that the capillary can draw fluid from the well. Capillaries on the output end 102 can be tightly bound and processed to form a two dimensional array. The minimum number of tubes may depend upon the number of probes to be deposited (e.g., 10³ to 10⁷).

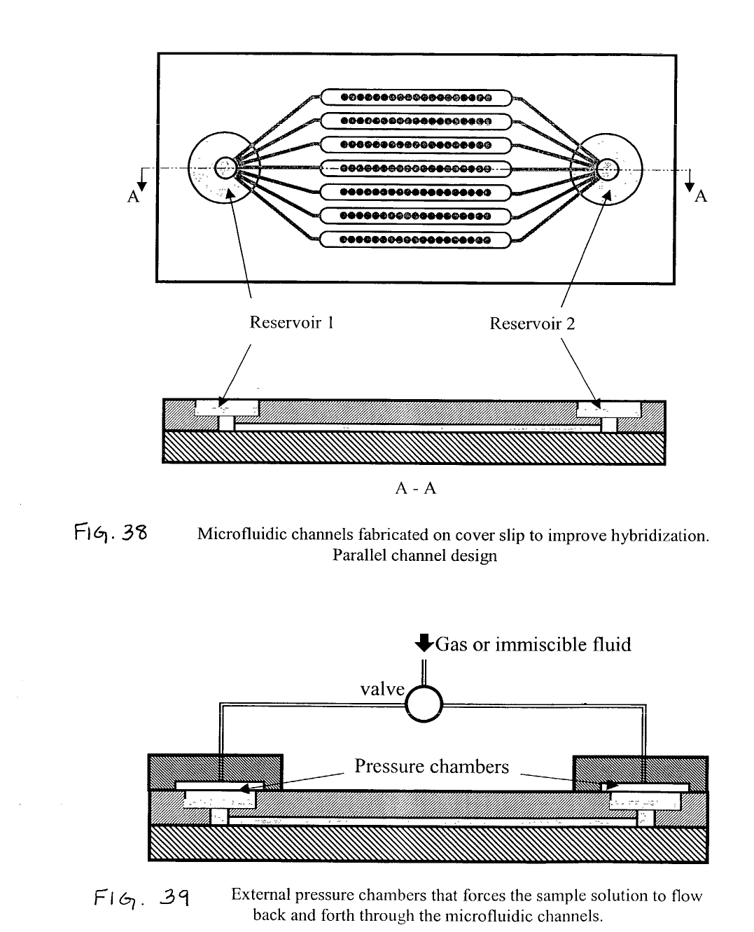

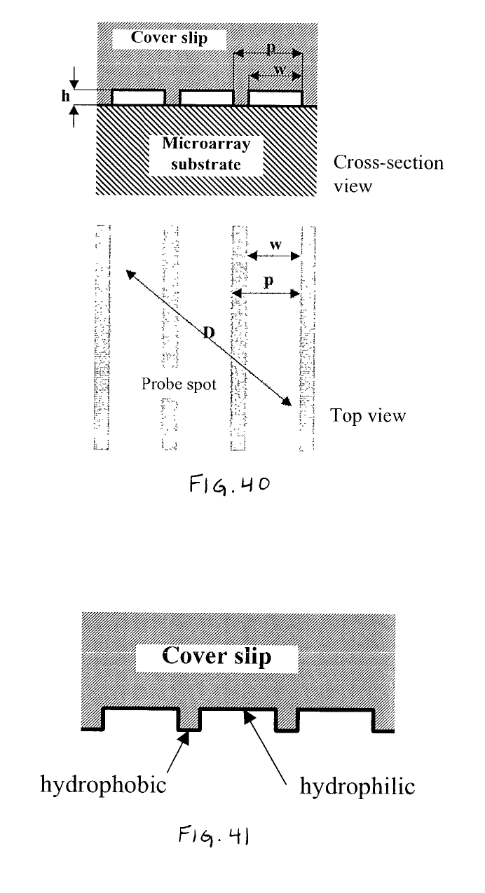

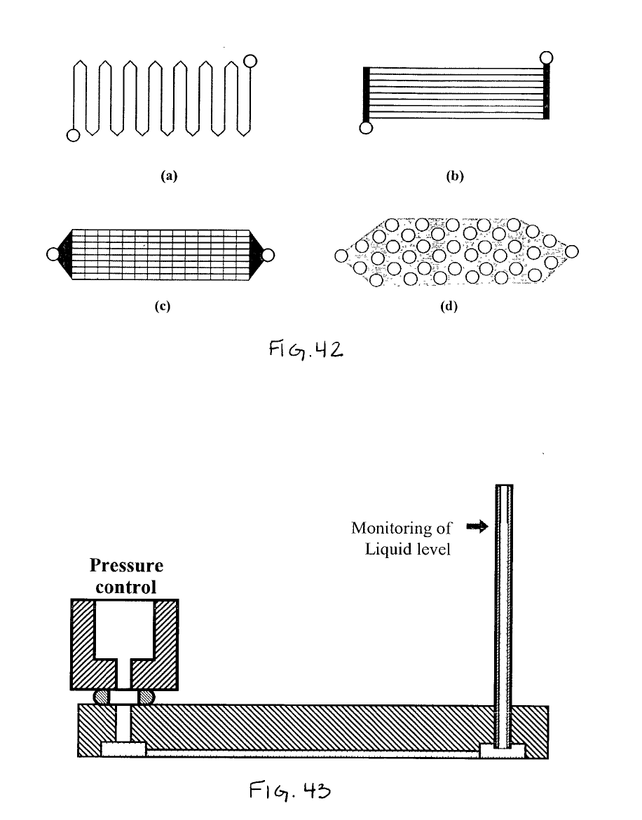

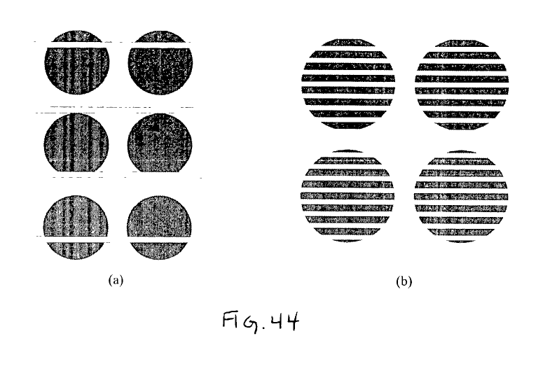

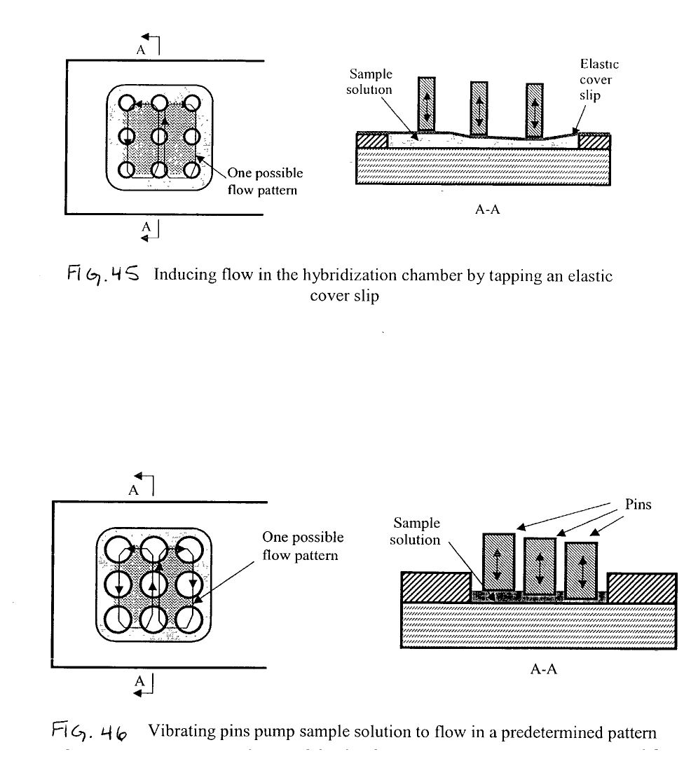

The probes can be delivered by applying pressure to the reservoirs (as illustrated in FIG. 4) or by gravity (as illustrated in FIG. 5) or by any of the other methods discussed in the pending U.S. and foreign patent applications noted above.

Numerous methods can be used to drive fluid from its reservoir into the capillary and towards the reaction chamber. These methods can be used alone or in combination of one or more other methods.

In one embodiment, a differential air pressure can be established and maintained between the proximal and distal ends of the capillary bundles, which will translate into hydraulic pressure to drive the probe fluids. FIG. 4 illustrates an embodiment of a pressure delivery system. One or more microtiter plates 210 are enclosed in a chamber 270. A probe 222 to be assayed is contained within each reservoir or well 220 of the microtiter plate 210. A free end of a capillary tube 100 connects to the well 220 such that it is in contact with the probe 222 which can be dispersed in a fluid form. Multiple such capillaries are bundled at an end 110 distal from the probes 222 to form delivery head 250.

In one embodiment, compressed air or an inert gas such as nitrogen 280 is pumped into a sealed chamber 270 carrying the microtiter plates. The probes 222 from microtiter plate 220 are forced by hydraulic pressure through the capillary tube to the print head 250.

In an alternative configuration, the output ends of the capillaries 100 may be placed under a vacuum or a lower pressure than the reservoirs 220. The print head 250 and substrate holder may be placed within a vacuum chamber, and the capillaries may extend through a wall of the vacuum chamber and to the reservoirs 220. By lowering the air pressure at the bundled delivery head 250 relative to the pressure at the input end, fluid can be drawn from the reservoirs 220 to the print head 250.

Once the capillaries are filled with the probe fluids, a constant flow can be maintained and controlled by adjusting the vertical positions of the fluid reservoirs, e.g. the microtiter plates, with respect to the position of the reaction chamber. In the gravity delivery system illustrated in FIG. 5, the chemical compounds 222 are dispersed in the wells of a microtiter plate 320. Capillaries 310 connect at the input end to the microtiter plate 320 and form a delivery head 300 at the output end. By positioning the microtiter plate at a height 340 above the head 300, differential gravitational force is used to siphon the chemical compound from the wells of the microtiter plate 320 to the end of the delivery head 300. The height differential may be transiently operated such that once the compound reaches the end of the reaction/delivery head 300, further flow is ceased by eliminating the height differential. Thus the flow of the chemical compound may be controlled merely by altering the height of the microtiter plate 320 relative to the reaction/delivery head 300.

Because fluids are negatively or positively charged, a voltage applied between the reservoir and the reaction chamber can be used to control the flow of the fluid through electrostatic and electro-osmotic force (EOF). A voltage source may be connected to an electrically-conductive material on a facet of the input end 102 and to an electrically conductive material contacting the probe-containing liquid near the output ends of the capillary tubes 100. A voltage regulator may be used to regulate the voltage and thus the rate of deposition of probe molecules.

Another aspect of the invention may have a bundled end, a plurality of reservoirs, and a magnetic field generator that is positioned sufficiently close to the bundled end to move a magnetic probe-containing fluid (such as a fluid containing magnetic beads or paramagnetic beads having probes optionally attached to their surfaces) through the capillaries of the bundle.

II. Probe Immobilization

Immobilization of probe molecules on the substrate is used in preparing a variety of array embodiments of this invention. Various methods for surface attachment chemistries can be used, as described below.

A. Protected-Aldehyde Silanization Agents

In conventional methods, a surface functionalized aldehyde slide having surface immobilized functional groups with terminal aldehyde groups for attachment of polynucleotides or other biomolecules is prepared in a two-step method consisting of immobilization of an aminoalkyl silane on a substrate to provide terminal amino groups, followed by conversion of the terminal amino groups with glutaraldehyde to terminal aldehyde groups. However, such conventional methods may result in numerous undesired defects and side products, including residual amino groups and unreactive condensation products.

In one embodiment of this invention shown in FIG. 6A, a protected aldehyde silane is prepared and used to functionalize a substrate in a one step silanization reaction. Substrates functionalized in this reaction have no residual amino groups, and substantially lack non-aldehyde by-products. An acetal compound comprising a protected aldehyde is prepared by hydrosilylation reaction of triethoxysilane with an alkenyl acetal. A variety of carbon numbers for the alkenyl group may be utilized, providing a variety of alkyl chains for use as a spacer between the silane group and the acetal group, including isomeric mixtures of alkyl chains. The spacer group may also be a polymer or chemical group. The protected aldehyde product may be immobilized on substrates such as glass slides in a one step silanization reaction. The resulting substrate is functionalized with protected aldehyde groups that may be deprotected to provide a surface functionalized by aldehyde groups. Alternatively, a non-protected aldehyde silane may be prepared by hydrosilylation reaction of triethoxysilane with an alkenyl aldehyde. The silane aldehyde may be utilized in combination with the protected aldehyde product to functionalize a substrate.

A substrate may be functionalized with the protected aldehyde silane by a variety of techniques. For example, solution phase reaction of the protected aldehyde silane with the substrate surface may be used. Alternatively, vapor phase deposition of the protected aldehyde silane on the substrate surface may be used. In another embodiment, the substrate is cured after reaction of the protected aldehyde silane with the substrate. Curing may be performed over a wide range of temperatures for a period as long as one day, or longer. These conditions and techniques are well-known to those in the field.

The protected aldehyde silane of the functionalized substrate may be deprotected by a variety of reactions to produce active aldehyde groups. Deprotection may be performed with, for example, trifluoroacetic acid or hydrochloric acid, among others, resulting in a reactive surface aldehyde slide. Such slides are useful for attachment of polynucleotides and other biomolecules, for example, having amino linking groups.

B. Maleimide Silanization Agents

Another composition and method for immobilization of reagents and molecules on the substrate are functional linker groups. In conventional methods, a surface functionalized slide is first prepared having attached functional linker groups with known ability to link, for example, polynucleotides or other biomolecules having various reactive groups such as amino groups, sulfhydryl groups, or phosphothionate groups. For example, an aminoalkyl silane is immobilized on a substrate to provide a surface having attached functional groups with terminal amino groups. In a second step, the functionalized substrate is reacted with a maleimide carboxylate to provide a reactive maleimide group attached to the surface linker group. The reactive maleimide groups are used to attach a polynucleotide. However, this conventional method typically results in undesirable residual amino groups.

In one embodiment of this invention shown in FIG. 6B, a maleimide silane is used to functionalize a substrate in a one step silanization reaction. In a maleimide silane, the reactive maleimide group is separated from the silane group by a spacer group which may have, for example, any one of a variety of carbon numbers to provide a variety of lengths of spacer chains between the two reactive groups. Substrates functionalized in this reaction have reactive maleimide groups immobilized on the surface, and no residual amino groups. The reactive maleimide groups on the surface may be reacted, for example, with sulfhydryl functionalized polynucleotides or other biomolecules to be attached to the surface. Unreacted maleimide groups may be blocked with various sulfhydryl-containing reagents, to provide a substrate with attached polynucleotides or other molecules, useful as probes. In further embodiments, the spacer may be one of a variety of polymers or chemical chains, for example, a polyethylene glycol. Various reagents may be added to the sulfhydryl functionalized reactant to prevent cross linking or other coupling of the molecules, such as a reagent to prevent disulfide bond formation.

A substrate may be functionalized in a one step silanization reaction with the maleimide silane by a variety of techniques. For example, solution phase reaction of the maleimide silane with the substrate surface may be used. Alternatively, vapor phase deposition of the maleimide silane on the substrate surface may be used. In further embodiments, the substrate may be cured after reaction of the maleimide silane with the surface. Curing may be performed over a wide range of temperatures for a period as long as one day or longer.

C. Light Activation of Arrays

In further embodiments, the substrate may be chemically functionalized with surface-immobilized protected functional groups, where the protected functional groups are capable of being activated by absorption of light to provide reactive activated functional groups. The activated functional groups may be used to attach molecules, cells, or biomolecules to the surface. A mask or fiber optic bundle may be used to create a substrate having interspersed regions of activated and non-activated functional groups by irradiation of the substrate with light through the mask or fiber optic capillary bundle. The size, features, and morphology of the regions having activated functional groups are precisely controlled by the mask or fiber optic bundle. Biomolecules may be delivered to the surface and react to bind to the activated functional groups. Thus, the surface can be patterned to provide regions with bound biomolecules of precisely controlled size and morphology, regardless of the size or features of the region where the biomolecules were initially delivered to the surface.

In one embodiment shown in FIG. 6C, an aldehyde silane as discussed previously is used to functionalize the substrate by a silanization reaction. The aldehyde silane includes a photoreactive or photolabile group which, upon irradiation of the substrate, is cleaved from the surface immobilized silane, leaving a reactive aldehyde group attached to the substrate. The photolytic reaction can also be controlled by introducing a solvent to the substrate surface, or, for example, by introducing one or more of various photosensitizer or photoinhibitor agents to the surface.

Other methods for binding biomolecules, such as polypeptides and proteins, nucleic acids, carbohydrates, lipids, and metabolic products or other ligands, as well as larger biological assemblies such as viruses, subcellular organelles, or even cells, to solid supports are well-known and characterized in the art. Generally, a biomolecule or other structure may be immobilized either covalently or non-covalently to the support; either type of binding may require modification of the biomolecule, or the support, or both. In some cases, a binding pair, such as avidin/streptavidin and biotin, is used and one member of the pair is linked to the solid support while the other is linked to the biomolecule.

For nucleic acids, there are many techniques available and in common use, including covalent immobilization with or without pretreatment of support and/or nucleic acid (see, e.g., U.S. Pat. Nos. 6,048,695; 5,641,630; 5,554,744; 5,514,785; 5,215,882; 5,024,933; 4,937,188; 4,818,681; 4,806,631; Running. J. A. et. al., BioTechniques 8:276-277 (1990); Newton, C. R. et al. Nucl. Acids Res. 21:1155-1162 (1993)), non-covalent immobilization (e.g., U.S. Pat. No. 5,610,287), immobilization via avidin/streptavidin-biotin (e.g., Holmstrom, K. et al., Anal. Biochem. 209:278-283 (1993)). One very common substance used to prepare a glass surface to receive a nucleic acid sample is poly-L-lysine. See, e.g., DeRisi, et al. Nature Genetics 14: 457 (1996); Shalon et al. Genome Res. 6: 639 (1996); and Schena, et al., Science 270: 467 (1995). Other types of pre-derivatized glass supports are commercially available (e.g., silylated microscope slides). See, e.g., Schena, et al., Proc. Natl. Acad. Sci. (USA) 93: 10614 (1996).

For proteins, general techniques may be found in Methods in Enzymology, Vol. 44 (Immobilized Enzymes Edited by Klaus Mosbach, 1977); Vol. 135 (Immobilized Enzymes and Cells, Part B, Edited by Klaus Mosbach, 1987); Vol. 102 (Hormone Action, Part G: Calmodulin and Calcium-Binding Proteins, Edited by Anthony R. Means and Bert W. O'Malley, 1983); Academic Press, New York. Methods of covalent binding of proteins to supports may be found in, e.g., U.S. Pat. No. 5,602,207 and Zhang and Tam, Thazolidine formation as a general and site-specific conjugation method for synthetic peptides and proteins, Anal. Biochem. 233: 87-93 (1996), Support and method for immobilizing polypeptides.

Methods developed for the binding of antibodies to glass supports are of use, not only to bind antibodies, but other proteins as well. See, e.g., U.S. Pat. No. 5,646,001; Bhatia et al., Use of thiol-terminal silanes and heterobifunctional crosslinkers for immobilization of antibodies on silica surfaces, Anal. Biochem 178:408-413 (1989); Yanofsky et al., High affinity type I interleukin 1 receptor antagonists discovered by screening recombinant peptide libraries, PNAS USA 93: 7381-7386 (1996); Narang et al., A displacement flow immunosensor for explosive detection using microcapillaries, Anal. Chem. 69:2779-2785 (1997); Shriver-Lake et al., Biosens. Bioelect. 12:1101-1106 (1997).

Carbohydrates may also be immobilized to a solid support, either to bind substances to the carbohydrate, or to immobilize another moiety (e.g., a protein) which is attached to the carbohydrate. See, e.g., U.S. Pat. No. 6,231,733, entitled “Immobilized Carbohydrate Biosensor”, to Nilsson et al. The immobilized carbohydrate moiety may itself be specific for another type of biomolecule or structure, such as a protein, virus or a cell. A review of useful binding carbohydrate sequences can be found in, e.g., Chemistry and Physics of Lipids, vol. 42, p. 153-172, 1986, and in Ann. Rev. Biochem., vol. 58, p. 309-350.

Methods for binding other biomolecules, as well as artificial molecules, substrates, ligands, and other molecules useful for binding biomolecules or biological substances of interest, depend on the nature of the substance to be bound and will be readily apparent to one of skill in the art. See, U.S. Pat. Nos. 5,817,470; 5,723,344; e.g., Weng et al., Proteomics 2:48-57 (2002); Zhou et al., Trends Biotechnol 10 (Suppl):S34-9 (2001); Mousses, et al., Curr Opin Chem Biol 6:97-101 (2002); Mirzabekov and Kolchinsky, Curr Opin Chem Biol 6:70-5 (2002); Reininger-Mack, Trends Biotechnol 20:56-61 (2002).

III. Probes and Target Molecules

The probes bound to the microarray substrate surface can be any type of molecule which binds or hybridizes with target molecules contained in the target liquid. The target molecules can be any type of molecule which binds or hybridizes with the immobilized probes. In various embodiments, a target molecule used in one assay can be immobilized on a substrate and used as a probe for another assay. Similarly, the probes used in one assay can be suspended in a fluid and used as a target molecule for another assay.

In accordance with various embodiments of the present invention, the probes can be, for example, deoxyribonucleic acids (DNA), ribonucleic acids (RNA), synthetic oligonucleotides, antibodies, proteins, peptides, lectins, modified polysaccharides, synthetic composite macromolecules, functionalized nanostructures, synthetic polymers, modified/blocked nucleotides/nucleosides, modified/blocked amino acids, fluorophores, chromophores, ligands, chelates, haptens and drug compounds. In some embodiments, the probes are polypeptides.

In particular embodiments, the biological target molecule is a polypeptide, a nucleic acid, a carbohydrate, a nucleoprotein, a glycopeptide or a glycolipid, preferably a polypeptide, which may be, for example, an enzyme, a hormone, a transcription factor, a receptor, a ligand for a receptor, a growth factor, an immunoglobulin, a steroid receptor, a nuclear protein, a signal transduction component, an allosteric enzyme regulator, and the like. The target molecule may comprise the chemically reactive group without prior modification of the target molecule or may be modified to comprise the chemically reactive group, for example, when a compound comprising the chemically reactive group is bound to the target molecule.

Other embodiments of the above described methods employ libraries of organic compounds which comprise aldehydes, ketones, oximes, hydrazones, semicarbazones, carbazides, primary amines, secondary amines, tertiary amines, N-substituted hydrazines, hydrazides, alcohols, ethers, thiols, thioethers, thioesters, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds and/or acid chlorides, preferably aldehydes, ketones, primary amines, secondary amines, alcohols, thioesters, disulfides, carboxylic acids, acetals, anilines, diols, amino alcohols and/or epoxides, most preferably aldehydes, ketones, primary amines, secondary amines and/or disulfides.

Biological target molecules that find use in embodiments of the present invention include all biological molecules to which a small organic molecule may bind and preferably include, for example, polypeptides, nucleic acids, including both DNA and RNA, carbohydrates, nucleoproteins, glycoproteins, glycolipids, and the like. The biological target molecules that find use herein may be obtained in a variety of ways, including but not limited to commercially, synthetically, recombinantly, from purification from a natural source of the biological target molecule, etc.

In one embodiment, the biological target molecule is a polypeptide. Polypeptides that find use herein as targets for binding to organic molecule ligands include virtually any peptide or protein that comprises two or more amino acids and which possesses or is capable of being modified to possess a chemically reactive group for binding to a small organic molecule. Polypeptides of interest finding use herein may be obtained commercially, recombinantly, synthetically, by purification from a natural source, or otherwise and, for the most part are proteins, particularly proteins associated with a specific human disease condition, such as cell surface and soluble receptor proteins, such as lymphocyte cell surface receptors, enzymes, such as proteases and thymidylate synthetase, steroid receptors, nuclear proteins, allosteric enzyme inhibitors, clotting factors, serine/threonine kinases and dephosphorylases, threonine kinases and dephosphorylases, bacterial enzymes, fungal enzymes and viral enzymes, signal transduction molecules, transcription factors, proteins associated with DNA and/or RNA synthesis or degradation, immunoglobulins, hormones, receptors for various cytokines including, for example, erythropoietin/EPO, granulocyte colony stimulating receptor, granulocyte macrophage colony stimulating receptor thrombopoietin (TPO), IL-2, IL-3, IL-4, IL-5, IL-6, IL-10, IL-11, IL-12, growth hormone, prolactin, human placental lactogen (LPL), CNTF, octostatin, various chemokines and their receptors such as RANTES, (regulated upon activation, normal T cell expressed and secreted MIP1-.alpha., IL-8, various ligands and receptors for tyrosine kinase such as insulin, insulin-like growth factor 1 (IGF-1), epidermal growth factor (EGF), heregulin-.alpha. and heregulin-.beta., vascular endqthelial growth factor (VEGF), placental growth factor (PLGF), tissue growth factors (TGF-.alpha. and TGF-.beta.), other hormones and receptors such as bone morphogenic factors, follicle stimulating hormone (FSH), and leutinizing hormone (LH), tissue necrosis factor (TNF), apoptosis factor-1 and -2 (AP-1 and AP-2), mdm2, and proteins and receptors that share 20% or more sequence identity to these.

The biological target molecule of interest can be chosen such that it possesses or is modified to possess a chemically reactive group which is capable of forming a covalent bond with members of a library of small organic molecules. For example, many biological target molecules naturally possess chemically reactive groups (for example, amine groups, thiol groups, aldehyde groups, ketone groups, alcohol groups and a host of other chemically reactive groups; see below) to which members of an organic molecule library may interact and covalently bond. In this regard, it is noted that polypeptides often have amino acids with chemically reactive side chains (e.g., cysteine, lysine, arginine, and the like). Additionally, synthetic technology presently allows the synthesis of biological target molecules using, for example, automated peptide or nucleic acid synthesizers, which possess chemically reactive groups at predetermined sites of interest. As such, a chemically reactive group may be synthetically introduced into the biological target molecule during automated synthesis.

Moreover, techniques well known in the art are available for modifying biological target molecules such that they possess a chemically reactive group at a site of interest which is capable of forming a covalent bond with a small organic molecule. In this regard, different biological molecules may be chemically modified (using a variety of commercially or otherwise available chemical reagents) or otherwise coupled, either covalently or non-covalently, to a compound that comprises both a group capable of linking to a site on the target molecule and a chemically reactive group such that the modified biological target molecule now possesses an available chemically reactive group at a site of interest. With regard to the latter, techniques for linking a compound comprising a chemically reactive group to a target biomolecule are well known in the art and may be routinely employed herein to obtain a modified biological target molecule which comprises a chemically reactive group at a site of interest.

IV. Microarray Hybridization

In accordance with embodiments of the present invention, systems and methods are provided for facilitating interactions between molecules bound to a microarray substrate surface and molecules in a target liquid. Various systems and methods described below may not be limited to hybridization processes, but can also be applicable for other molecular interactions, such as, for example, associations, complexing, reactions, ionic and/or hydrogen bonding, bonding between molecules.

To minimize consumption of sample fluid, the hybridization chambers in existing hybridization systems are normally several centimeters across in the XY plane but tens of micrometers in thickness (Z). Liquids contained in such a chamber may exhibit typical microfluidic behavior because the small Z dimension causes the surface to be tension dominant. If no flow is introduced in the chamber, the liquid-probe mixing can only be achieved through diffusion, which is very slow and practically impossible across such a large XY dimension. Because of this, each probe only hybridizes with target molecules in a small volume near the probe in a “static hybridization” condition, which significantly reduces the detection sensitivity. To improve the sensitivity, a “dynamic hybridization” condition can be created where the sample liquid is driven to mix thoroughly with the probe array.

A. Hybridization Apparatus with Movable Substrate or Cover

The rate of hybridization can be increased by introducing active mixing during hybridization by creating relative motion between a substrate and a cover of a hybridization apparatus. An array hybridization apparatus incorporating a movable substrate or a movable cover includes a substrate and a cover, wherein the substrate and/or the cover are movable relative to each other. The substrate can be in the form of a flat substrate slide on which an array of probes is deposited. The cover can be a cover slip which mates with the substrate slide to form a hybridization chamber.

1. Target Liquid Confinement

A target liquid added to an array hybridization apparatus between a substrate slide and a cover slip may be confined by using a surface tension differential created on the surface of the substrate slide and/or the cover slip. The surface of the substrate slide can have a coating to form a hydrophilic region surrounded by hydrophobic region. The hydrophilic region contains an array of probes. Surface energies between the hydrophobic and hydrophilic coating confine the target liquid within the hydrophilic region. The substrate slide can be designed to have multiple hydrophilic regions separated or surrounded by hydrophobic regions so that multiple liquid samples and multiple probe arrays can be applied to the same substrate slide without cross-contamination. In other embodiments, the target liquid can be contained on the substrate using a hydrophobic region surrounding an untreated region. Similarly, the target liquid can be contained on a hydrophilic region surrounded by an untreated region.

As used herein, the term hydrophobic is used to describe a surface or coating which forms a contact angle of greater than 90° when a droplet of water is deposited thereon. The term hydrophilic is used to describe a surface or coating which forms a contact angle of less than 90° when a droplet of water is deposited thereon.

Numerous methods are available for forming the hydrophilic and hydrophobic coatings or materials used in embodiments of the present invention. For example, various methods are described in U.S. patent application Ser. No. 10/080,274, entitled “Method and Apparatus Based on Bundled Capillaries for High Throughput Screening,” by Shiping Chen et al., filed Feb. 19, 2002, incorporated by reference herein in its entirety.

In accordance with one embodiment, masking technology is utilized to prepare localized areas on the surface for selective hydrophilization. FIG. 7 shows a process for fabrication using a negative mask. In this method, the entire surface of a substrate is first functionalized with a hydrophobic (“1”) chemistry. Next a mask is placed on the substrate surface and the hydrophobic chemistry is removed from the exposed regions using, e.g., a chemical removal process. The exposed (and stripped) regions are then functionalized with a hydrophilic chemistry (“2”). The localized hydrophilic regions can alternatively be formed using a positive masking process.

Techniques of ultraviolet (UV) ablation may also be used in surface tension patterning embodiments. The substrate is functionalized or coated with ablatable material or molecules. UV radiation is used to selectively ablate the coating from regions of the substrate by using a mask, thereby patterning the substrate. Regions from which ablatable material was removed may be further functionalized to create a pattern of interspersed regions of differing surface tension.

FIG. 8 shows that if two different samples are to be analyzed then one sample is placed in region 1 and another sample is placed in region 2. Areas on the substrate labeled “1” are hydrophobic. Areas on the substrate labeled “2” are hydrophilic. Probe polynucleotide strands are immobilized on the hydrophilic regions of the substrate surface. Liquid droplets comprising potential targets for the probe ararys are localized to the probe regions by surface tension. An anchored cover slip is added to control dispersion of the target liquid. The interaction between the target liquid and the immobilized probes may then be promoted by agitating the substrate slide. Alternately the cover slip itself may be rotated or agitated, optionally by electromagnetic means, to agitate the solution and ensure movement of the target liquid as described below. The surface tension characteristic of the substrate slide inhibits the droplet from dispersing even in light of the relative movements of the substrate slide and the cover slip. The relative movements of the substrate slide and the cover slip can be adjusted to generate less force than the surface tension holding the target liquid on the substrate slide.

The sample solution can also be further confined by surface tension differential on a cover slip surface. The cover slip can be coated with uniform hydrophobic coating so that the hydrophobic coating enhances the surface tension that holds the liquid sample underneath. The cover slip can also be coated with confined hydrophilic regions surrounded by hydrophobic regions that match the hydrophilic regions on the substrate slide. In this design, an area that is the same size and shape of the sample area on the substrate slide is made hydrophilic, while the area outside is made hydrophobic. The patterning of both the slide and the cover slip will further assist in confining the solution to the sample area. In addition, the hydrophilic area will pull the solution with it during agitation, thus create more effective movement of the sample solution.

2. Cover

In accordance with embodiments of the present invention, a cover is coupled with the substrate to contain the target liquid therebetween. The cover can serve multiple functions. First, it can be used to minimize evaporation of a liquid target sample by reducing the exposure of the target liquid to the environment. Second, by compressing the target liquid, a small amount of target liquid can be spread out to cover a larger probe array area. Finally, the cover can be used to generate movement of the target liquid and thereby promote interaction between the target liquid and the probes on the substrate. The movement of the target liquid can be accomplished by causing relative movement between the cover and the substrate.

Numerous cover slip designs can be used for the purpose of liquid confinement and movement. In a first example, the cover slip surface may have a uniform hydrophobic coating. The hydrophobic coating of the cover slip enhances the effect of the surface tension differentials on the substrate slide for holding a hydrophilic target liquid within the hydrophilic region on the substrate slide. In a second example, the cover slip surface may have a coating with one or more confined hydrophilic regions surrounded by hydrophobic regions that match the hydrophilic/hydrophobic pattern on the substrate slide. In the second example, the patterning of both the substrate slide and the cover slip can further enforce the confinement of a hydrophilic target liquid to the hydrophilic region. In addition, the hydrophilic area will pull the target liquid with it during agitation, thus creating more effective movement of the target liquid. Alternatively, if the target liquid is hydrophobic, the cover slip surface and the substrate surface may have a coating with one or more confined hydrophobic regions surrounded by hydrophilic regions wherein the hydrophobic/hydrophilic pattern on the cover slip matches that on the substrate slide.

In some embodiments, the cover can be moved by a force, such as magnetic and mechanical force. In addition, to increase the effectiveness of movement of target molecules, protrusions can be engineered on the surface of the cover facing the target liquid. The cover may also have risers which form a container slightly larger than the substrate so that the substrate can be inserted into the cover container during hybridization.

In the embodiment illustrated in FIG. 9, the cover slip is magnetized, contains magnetized components, or contains magnetically reactive components. This magnetized cover slip can be made by attaching a magnet to a typical glass cover slip or by forming the cover slip out of magnetic glass. A support fixture may be provided to align the cover slip with the substrate slide and to prevent the cover slip from falling off the substrate slide. An example of the support fixture is shown in FIG. 9. This assembly can be placed on a magnetic stirring table similar to a hot plate stirrer commonly used in laboratories. The magnetic driver under the table generates a moving magnetic field, which in turn drives the magnetic cover slip to rotate or move in a circular motion. The motion of the cover slip induces flow and turbulence in the sample liquid sandwiched between the cover slip and the substrate slide, which can enhance the interaction between sample liquid and the probes on the substrate slide.

In another embodiment, certain surface textures can be engineered on to the surface of the cover slip that is in contact with the sample liquid. This can enhance the capability of the cover slip to induce flow in the sample liquid. The technique can be particularly effective when the target liquid is confined by surface tension differential on either the microarray or cover slip surface. The cover slip should rotate fast enough to generate movement for efficient hybridization but not so fast as to disrupt interactions between target molecules in the sample liquid and probes on the substrate. Alternatively, the cover slip can be agitated at high speeds to enhance mixing, and then slowed or stopped to enable effective interactions.

In another embodiment, effective movement of the liquid sample can be created using a floating and sliding cover slip. This method combines a rigid cover slip that permits low volumes of target liquids with mechanical movements to achieve dynamic movement of the target liquid. This design may incorporate the hydrophobic/hydrophilic surface tensions described above to retain liquid between the cover slip and the substrate slide. The substrate slide is patterned so that the area that contains probes of interest, such as DNA probes, is hydrophilic, while the surrounding areas are hydrophobic.

In the absence of the cover slip, aqueous solution applied to the substrate slide will form a droplet on top of the hydrophilic area with a contact angle determined by the hydrophobicity of the surrounding area (shown in Side View 1, FIG. 10). When a cover slip is positioned on top of the droplet, a smaller volume of liquid is required to fill the same-sized hydrophilic area (Side View 2, FIG. 10). Due to the surface tension, the aqueous solution will be confined within the hydrophilic region, and the cover slip can be supported by the aqueous solution, thereby “floating” on top of the droplet.

The cover slip coupled with the microarray substrate forms an assembly which in some embodiments can be placed in a slide holder. The slide holder can serve to seal the assembly to inhibit evaporation and limit the movement of the substrate slide or the cover slip.

Various assembly designs can be used to generate relative motion between the cover and the substrate. In FIG. 12, an immobilized substrate slide (2) having a plurality of probe arrays (4) is confined by barriers (7) on the substrate holder (1) with relatively little room for movement. On the other hand, the cover slip (5) which floats on top the liquid sample is loosely retained by barriers (8) on the cover holder (6). Because the barriers (8) on the cover holder (6) provide some lateral clearance for the cover slip (5), the cover slip (5) can move laterally over a relatively larger area within the cover holder (6). A barcode (3) can be provided on the substrate slide (2) to facilitate handling and organization of the substrates.

The barriers (8) are engineered on the cover holder (6) so that when the entire assembly is agitated, the cover (5) will slide to one side until it hits the barrier (8). Agitating the assembly in multiple directions will result in the continuous movement of the cover slip, thus generating movement of the target liquid underneath. This sliding motion provides agitation to move the target molecules of the sample liquid to facilitate better binding with the probes in the microarray.

The barriers (7) and (8) can take various forms. In some embodiments, a single barrier encircles the entire substrate (2) or cover (5). In other embodiments, a plurality of smaller barriers are used to limit the movement of the substrate (2) or cover (5) in at least one direction.

In an alternative embodiment, the cover (5) can be immobilized in the cover holder (6). In this design, the cover (5), rather than the substrate (2), is confined by barriers on the cover holder (6). Confinement barriers in the substrate holder (1) will provide increased lateral clearance so that the substrate (2) will be able to move laterally for a limited distance.

In another embodiment, the cover slip may have protrusions or ridges to enhance the agitation of the target liquid and generate more effective movement of the target liquid underneath. For example, the protrusions can be formed as tooth-like ridges such as the design shown in FIG. 11.

In FIG. 11, the cover slip is fabricated to have tooth-like structures on the surface that contact the target liquid. Each of these teeth are formed as a ridge with a front side that is aligned roughly perpendicular to the surface of the substrate (a 90° angle) and a back side that is at less than a 90° angle to the substrate surface. Because of the shape of these teeth, the liquid is “pumped” to flow preferentially in one direction when the cover slip moves vertically up and down.

A small rocking motion can be introduced into the vibration to enhance the pumping action, as shown in FIG. 11. This can be achieved by attaching a PZT on the cover or placing the substrate/cover assembly on a vibration table designed for supporting the substrate while moving the cover slip. The cover slip is driven to move up and down against the substrate slide by an acceleration force generated by generated by the PZT or some other motion or vibration inducing device.

In some embodiments, the orientation of the ridges changes direction on opposite edges of the cover slip. In this way, a rotational flow pattern can be established when the cover slip is moved in a circulating motion relative to the microarray substrate slide, as shown in FIG. 11 to generate a circular flow in the target liquid.

3. Substrate

Hybridization can also be promoted by introducing active movement of a target liquid during hybridization by mechanically moving the microarray substrate or substrate slide. As illustrated in FIG. 13, instead of introducing target liquid onto a microarray substrate slide as in conventional hybridization devices, a microarray substrate slide (shown in FIG. 13 as microarray carrier) can be inserted into a cover slip having a reservoir containing the target liquid. Lateral and rotational movement can be introduced to the microarray substrate slide to encourage interactions between the target liquid and probes. For example, the slide and/or cover slip can be mounted in movable stages that impart lateral and/or rotational movements.

In some embodiments, the size of the sample liquid container is slightly larger than that of the microarray substrate slide to minimize the volume of target fluid used to cover the entire surface of the microarray. In the embodiment shown in FIG. 13, the microarray substrate slide is not a standard microscope slide. Instead, the substrate is shown as a cylindrical microarray carrier having the probe microarray deposited on one end. In another embodiment, the microarray substrate slide is mounted to the facet of a rotating member, such as a short pole, and the sample solution is contained in the well of a standard microtiter plate. Multiple samples can be hybridized to multiple microarrays in parallel, but coupling multiple substrates with the multiple wells in the microtiter plate.

B. Fixed Substrate Slide and Cover Slip Hybridization Apparatus

An embodiment of a hybridization apparatus includes a cover slip formed with a very flat surface and with spacers provided on the outer edges of the slip. The height of the spacer can be precisely controlled using precision fabrication techniques, such as etching or electroplating. By forming the slip with an extremely flat surface and precisely-fabricated spacers, the thickness of the target fluid across the probe array can be highly uniformly controlled. This can improve the uniformity of the hybridization across the microarray.

An embodiment of a hybridization apparatus includes a hybridization assembly and a target liquid motion inducer in a hybridization chamber. The hybridization assembly comprises a reaction chamber (or hybridization chamber) to confine and allow interaction or binding of a target liquid to an array of probes deposited on an inner surface of the reaction chamber. The hybridization assembly may comprise a substrate slide, a gasket layer and/or a middle slide, and a cover slip. The cover slip, the gasket layer and/or the middle slide, and the substrate slide can be fastened together to form a watertight hybridization chamber. FIG. 14 shows an example of a “sandwich” hybridization chamber. For hybridization, the chamber is filled with the target liquid. The substrate slide has an array of probes deposited on the substrate slide surface facing the hybridization chamber. A spring steel slide holder or other clamping mechanism may be used to maintain pressure in the slide stack, as shown in FIG. 15.

The middle slide has a through opening which can be precisely formed to be slightly larger than the outer dimensions of an array of probes on the substrate slide so that the array of probes is positioned inside the opening when the middle slide is placed on the substrate slide. Alternatively, the middle slide may have a plurality of openings that match a plurality of arrays of probes on the substrate slide. The thickness of the middle slide may be, for example, from 10 μm to 5 mm. The substrate slide and the middle slide can be made of any suitable materials including glass, silicon, polymer, plastic, ceramic, metal, wood, rubber, silicone rubber, etc.

When the middle slide is made of a relatively hard material such as glass, ceramic or metal, a gasket layer may be attached to the surface of the middle slide that contacts the substrate to serve as a seal (FIG. 16). This gasket layer should ideally be made of softer and hydrophobic material such as silicone rubber, polytetrafluoroethylene, Teflon®, or polydimethylsiloxane (PDMS). The method of attachment can be lamination, injection molding gluing or any other means, or the gasket layer can be held in place by the clamping force. If both the substrate and the middle slide are very flat, it is also possible to make the gap between the middle and substrate slide surfaces water tight by simply making both surfaces highly hydrophobic and pressing the two tightly together.

During hybridization, the middle slide is placed on the substrate slide with the array of probes positioned inside the opening. The middle and substrate slides are tightly pressed against each other to provide a watertight seal preventing fluid leakage through a gap between the two slides. Atmospheric pressure is often sufficient to maintain the seal. For added assurance, a spring slide holder designed to clamp the slides together by applying pressure to the outer surfaces can be used to maintain the pressure, as illustrated in FIG. 15. In this way, the opening through the middle slide and the substrate forms wells on the microarray into which one or more sample or target liquids are introduced using a precision liquid delivery device such as a pipette.

The volume of the sample liquid may be controlled so that the liquid surface in the “wells” created by the middle slide and the gasket layer (as illustrated in FIG. 14) is below the upper surface of the middle slide. Because both the volume of the sample liquid and the dimension of the middle slide opening can be precisely controlled, the height of the liquid inside the well, and thus the effective target hybridization volume can be precisely metered. In this way, the chip-to-chip hybridization variation can be minimized. A cover slip can be placed on top of the middle slide to reduce evaporation.

For another embodiment of this device, the cover slip and the middle slide can be an integrated piece, as shown in FIG. 17. The integrated cover slip has a well that is slightly larger than the outer dimensions of an array of probes on the substrate slide. When the cover slip is aligned with the microarray substrate slide, the well covers the array of probes on the substrate slide. The cover slip may have a plurality of wells that match a plurality of arrays of probes on the substrate slide. The cover slip can be made of, for example, plastic, polymer, glass, silicon, metal, ceramic, wood, rubber, silicone rubber, or any other suitable materials. The wells can be formed by machining, etching, molding or other suitable processes. A very thin gasket layer can be bonded to the lower surface of the integrated cover slip, which provides a seal at the interface between the integrated cover slip and the substrate slide.

In another embodiment shown in FIG. 18, the cover slip can be flat and have a thick gasket layer bonded to the bottom surface. The gasket layer has openings which form the wells.

In various embodiments, during hybridization, the cover slip is placed upside down and such that the wells face up, as shown in FIG. 19. Sample or target liquid is added to the wells (FIG. 19a). Then the microarray substrate slide is placed upside down on the cover slip, i.e. the surface having the microarray probes deposited thereon faces the cover slip. The cover slip and the substrate slide can be pressed tightly against each other to squeeze out air bubbles from the interface between the slide and the gasket (FIG. 19b). Before hybridization, the entire assembly is inverted to position the microarray substrate underneath the cover, thereby allowing the target fluid to contact the array of probes, as shown in FIG. 19c. A spring clamp or steel slide holder similar to the one illustrated in FIG. 15 can be used here to maintain pressure between the cover slip and the substrate slide.

In another embodiment shown in FIG. 20, the cover slip is a layer of liquid deposited over the sample solution, thereby forming a “lid” or layer to prevent evaporation of the sample liquid. This liquid layer can be selected to be immiscible and non-reactive with the sample solution. The liquid cover layer can also be deposited while in liquid form, and hardened into solid or semi-solid form after deposition to form the “lid.”

In some embodiments, the cover slip and the middle slide can be single use consumables or they can be reused for many different hybridizations after washing.

The movement of the target liquid can be created by forces such as, for example gravity, centrifuge force, magnetic force, sonic force, electronic force, Lorentz force, thermodynamic force, pneumatic force, or/and mechanical force, as described in greater detail below.

To generate effective motion in the hybridization chamber, a certain amount of “volume exclusion” (VE) liquid may be added to the hybridization chamber together with the target liquid. The VE liquid may be selected to have one or more of the following characteristics: inert, i.e., no adverse effects on dyes and probes; immiscible with the target liquid; lighter or heavier than the target liquid; and having a contact angle similar to that of the target liquid on the substrate slide. For example, one VE liquid which may be used is mineral oil.

Unlike an entrapped air bubble, the VE liquid can be selected to have similar surface tension characteristics as the target liquid. This can make it easier to move the interface between these two liquids in the chamber and to create relative movement between the two liquids. FIG. 21 illustrates the circulation of both VE and sample liquid in the chamber when the assembly is rotated in the presence of a gravitational field. In the embodiment shown, the VE liquid is less dense than the target liquid. As the assembly is rotated, gravity will draw the more dense target liquid to the bottom of the chamber, thereby displacing the VE. This movement of the target liquid can improve the circulation and mixing of the target liquid.

When the contact angles of the chosen VE liquid and target liquid are substantially different, the interface of the two liquids may increase the difficulty of causing relative movement of the two liquids using the force of gravity alone. In other cases, it may be desirable to increase the circulation of target liquid beyond the circulation provided simply through the use of gravity and rotation. In these situations, a number of methods can be used to force the VE liquid to move relative to the target liquid. A first method is to put the assembly in a centrifuge. The centrifugal force provides many times the force of gravity to move the VE and the target liquid. A second method is to use a magnetized liquid as described below.

FIG. 22 illustrates the use of magnetic forces to generate effective movement of target molecules in the hybridization chamber. In one embodiment, magnetic or magnetically reactive particles of various shapes can be added to the target liquid. A varying magnetic field can be generated in the solution to drive the particles moving in either a random or a pre-defined pattern. This moving magnetic field will cause the sample solution to flow in the same pattern. The surface of the magnetic particles can be coated so that the target molecules in the sample solution will not attach to the particles.

The varying magnetic field can be generated, for example, by using multiple magnetic pins positioned in a designated spatial pattern, such as the pattern shown in FIG. 22. A large magnet positioned under the microarray substrate can be switched on periodically to induce flow in the vertical direction. In FIG. 22, the pins are placed above the sample solution on top of the cover slip. The pin array can also be positioned below the microarray substrate, formed as part of the cover slip or the substrate, or even dipped into the sample solution when there is no cover slip present. Electric coils wrapped around the pins are energized to selectively magnetize certain pins in either a random or a designated timing and sequence. As shown in FIG. 22, the use of a designated magnetization timing and sequence can induce a flow pattern in the target fluid. In a simpler configuration, rotational magnetic fields can be generated in the sample solution by placing a coil set commonly used in electric motors under the microarray substrate. In yet other embodiments, varying magnetic fields can be generated to induce turbulent flow of the sample solution.

To avoid the magnetic particles from scratching the probes on the substrate slide when the particles are attracted to the microarray surface due to magnetic forces, the microarray-cover slip assembly can be flipped and the cover slip positioned closer to the magnetic source. Alternatively, two separate magnetic sources above and below the microarray-cover slip assembly can be used, as illustrated in FIG. 23. Each magnetic source generates a magnetic field that moves in the same direction. They are switched on and off in turn. In this way, the particles will follow a zig-zag path bouncing between the substrate and the cover, which induces the liquid sample to flow in the same fashion.

Magnetic volume exclusion (VE) liquid may also be used to generate effective movement of target molecules during hybridization. Suitable magnetic liquids include ferrofluids and magnetorheological (MR) liquids. Ferrofluids are stable colloidal suspensions of single domain particles of ferromagnetic or ferrimagnetic materials. They have existed for more than sixty years but the concentrated liquids that are used today first appeared in 1965. Ferrofluids are formed of very small magnetic particles held in suspension in a carrier liquid by a surface active layer. The carrier liquid is selected to meet the particular application and can be, for example, a hydrocarbon, ester, perfluoropolyether, water, or other liquid compatible with the target and probe molecules.

In this embodiment, the carrier liquid of the magnetic particles should be immiscible in the target liquids. By applying a magnetic field near certain parts of the hybridization chamber, the MR VE liquid will be attracted to the magnet, as shown in FIG. 24. Moving the magnetic field in a circular fashion will drive the VE liquid to move along the same route and generate circulative flow in the sample liquid.

FIG. 25 illustrates a system in which acoustic or ultrasonic waves are applied to the surface of the cover and/or the substrate to generate surface waves to move the target liquid around the reaction chamber. The power and the frequency of the waveform synthesizer are selected so that the target molecules such as DNA/RNA molecules or the hybridized complex between the target molecules and the probes are not destroyed by the sound waves, yet the target liquid is still moved effectively. The transducer can be, for example, one of the following: PZT, loudspeaker, or any electrical energy to acoustic energy converter.

Because many biochemical molecules bear an electric charge, electric voltages can be used to drive a target molecule in the liquid sample to move toward and hybridize with its complementary probe in the microarray. FIG. 26 illustrates a specific configuration of such a hybridization apparatus. In this system, an electrode is positioned adjacent to the microarray substrate. Multiple electrode pads are provided on the cover slip. The cover slip can be made of, for example, silicon, glass, ceramic or any other suitable material. The electrode pads can be fabricated using, for example, the microfabrication technologies widely used in the semiconductor industry. These electrode pads can be provided on an outside surface of the cover slide or can be integrated into the cover slip. The voltage differential between each pad on the cover slip and the electrode under the substrate can be individually controlled by computer. If the target molecule is negative charged, the target will be propelled by a negative electrode and attracted to the positive electrode.

The adjacent electrode pads on the cover slip can be turned positive or negative with reference to the electrode under the substrate in a programmed sequence. For example, as illustrated in FIG. 27, a target molecule with a negative charge is initially positioned under Pad 1 on top of a first probe. When Pad 2 is given a positive charge, the negatively-charged target molecule is pulled towards Pad 2. Next, a negative change is applied to all of the Pads 1-7 for a period of time. This causes the target molecule to be driven towards the substrate surface under Pad 2, where a second probe is located. By this process, the target molecule is moved laterally by one pad-distance. When Pad 3 is turned positive and then negative, the target molecule is moved one step further to be positioned next to a third probe.

In this way, charged target molecules in the sample can be driven up and down between the cover slip and the substrate slide and are transported along the pads in a “zig-zag” fashion as illustrated in FIG. 27. This “zig-zag” movement is characterized by a change in direction of the moving charged particles of less than 180°.

The lower half of FIG. 27 illustrates the voltage distributions across the electrode pads in time sequence for achieving such transport effect. The frequency of the positive-negative change on the electrode pad is adjusted so that the target molecule can associate or hybridize with its complementary probe for a desired time before it is pulled away from the substrate surface. By programming the timing and/or voltages of the pad array across the entire cover slip, the system can drive target molecules to move along a predetermined route to contact each probe in a speedy and orderly fashion, as illustrated in FIG. 27.

An alternative voltage sequence is illustrated in FIG. 28. Pad 1 is given a positive charge first, which lifts the target molecule up (if it is not specifically hybridized to the probe). Then Pad 2 is turned positive and Pad 1 is turned negative. This moves the molecule to a new position just under Pad 2. When the entire pad array is then turned negative, the molecule is pushed towards the substrate surface under Pad 2. Now the molecule has advanced by one pad-position laterally. By repeating in this fashion, the target molecule in the liquid sample can be transported along a predetermined route under the electrode pads to contact each of the probes in the probe array.

This hybridization apparatus can significantly improve the rate and the sensitivity of microarray hybridization. First, the rate of hybridization is increased by increasing the chance that the target molecule collides with its complementary sequence because the target molecule is moved along the surface of the substrate in the hybridization chamber. Second, when the electrodes on the cover slip are positive, target molecules that are not specifically hybridized to a specific probe can be forced by the electric field to move away from the microarray. The voltage used is high enough to pull the unhybridized target molecules away from the probe without pulling away hybridized target molecules or any probe on the substrate slide. This action can enhance the hybridization specificity.

In FIGS. 27-29, all electrodes are isolated from the liquid sample. The transportation process can therefore be defined as a “dielectrophoresis” mechanism. This kind of electric transport system may utilize a relatively large voltage to transport charged particles. This is because the buffer solutions are relatively good conductors in comparison with conventional microarray substrates and cover slips, which are made of glass or other dielectric materials.

It is also possible to submerge a set of electrodes in the sample solution and make use of an electrophoresis mechanism to transport the target molecules. The spatial pattern of electrode pads can be the same as the system shown in FIG. 26 except the electrode pads are now provided on the surface of the cover slip that faces the substrate. An advantage of such a pad array configuration is that it is easier to set up a continuous circulating transport route and while utilizing a relatively lower voltage.

It is noted that increasing the density of pad arrays increases the number of electronic connections used and can increase the complexity of the flow control algorithm. FIG. 29 shows a simplified electrode configuration in which the electrodes are positioned near the sides of the hybridization chamber. It is possible to fabricate these electrodes by electric plating methods and combine the electrode pads with the risers on the cover slip. In this configuration, the electrodes are substantially thick such that they also function as spacers between the substrate and the cover slip.

In yet another embodiment, the upper electrode pads can be provided on the inner surface of the cover slip, as shown in FIG. 30. This can enable the target molecules to be transported in a lateral direction using a relatively smaller voltage. The electrode pads can be in direct contact with the sample solution (electrophoresis) or a very thin layer of dielectric material can be coated on the pads to provide isolation (dielectrophoresis). To create more vertical movement of the target molecules towards the probes on the substrate surface, the gap between the cover slip and the substrate can be formed as small as possible also shown in FIG. 30. Using, for example, precision etching as is found in semiconductor manufacturing, it is possible to form a gap having a height in the sub-micrometer range. Because of the small gap, the target molecules can reach the probes by diffusion relatively quickly.

Another way to create more movement of the target molecules towards the probes on the substrate surface is to coat a layer of a conductor, such as metal, on a conventional substrate to serve as the lower electrode, as shown in FIG. 31. If the selected conductive layer is not compatible with the probe or target liquid, a thin biocompatible layer can be coated on top of the conductive layer to provide a base for probe bonding and target hybridization. The biocompatible layer can be, for example, silicon dioxide, silicon, or any other suitable material. Alternatively, a suitable conductive material can be used as the microarray substrate so that the substrate itself can be used as the lower electrode. Examples of such materials include p or n type doped silicon. Alternatively, the substrate can be intrinsic silicon having an upper surface doped to become p or n type conductive layer to serve as the lower electrode.

FIG. 32 shows an alternative approach. In an electric field, there exists field lines which plot the direction of dielectric force in the field. Charged molecules are transported along these lines. By arranging two electrode pads of opposite polarity separated by a suitable distance, the curve of electric field lines will reach the substrate surface thus transporting target molecules not only horizontally but also vertically towards the probe on the substrate surface. Additional pads can be positioned between the two opposing electrodes. Switching sequences can be employed to ensure that the target molecules pass every probe on the substrate.

It is also possible to mix liquid crystals (LC) into the sample solution. Because LC are highly polar and highly elliptically shaped particles, they can easily be manipulated by external electric fields to move in desired directions along the field lines. As the LC are moved, the LC create a flow in the surrounding liquid, thereby moving the liquid more readily to bring target molecules in contact with probe molecules.

FIG. 33 illustrates an electric field gradient which can be used to drive negatively charged molecules in a liquid sample. The liquid sample can be, for example, an aqueous solution that is polar. When a negatively charged molecule, such as DNA or RNA, is subjected to an electrical field E, a dipole moment, P, is induced. By applying an inhomogeneous electric field to the dipole, the dipole will be forced toward the lower energy density region. Therefore, by applying an electrical field to the hybridization chamber such that the lower energy density region is along the surface of the substrate, the negatively charged molecules are forced towards the surface of the substrate. The hybridization process can be accelerated due to the higher possibility of collision between the target DNA/RNA molecules and the probes on the substrate.

FIG. 34 illustrates an embodiment in which Lorentz forces are applied to move charged molecules in the liquid sample. The spacers along the sides of the hybridization chamber can be formed to conduct electricity. This can be accomplished, for example, by forming metal coated areas on the cover, the substrate, or a middle layer at each side of the hybridization chamber to serve as spacers as well as electrodes. A voltage applied across the two electrodes drives charged target molecules in the hybridization liquid to move in parallel with the substrate surface. A pair of magnets establishes a magnetic field across the hybridization chamber in perpendicular to the motion of the charged molecules. The magnetic vector is oriented so that the Lorentz force will push the target molecules to migrate towards the probes on the substrate surface.

In one embodiment, the voltage can be held constant while the orientation of the magnetic field vector is periodically reversed. The Lorentz force reverses directions periodically causing the charged molecules to follow a zig-zag route between the cover slip and the surface of the microarray from one electrode to the other. The polarity of the voltage can also be switched to change the direction of the molecules movement. This can create improved contact between the target molecule and the probes on the substrate.

It is possible to split the two electrodes on each side or add two additional electrodes on the other two sides of the hybridization chamber, as shown in FIG. 35a and b, which show a top view of two embodiments of the invention. In FIG. 35a, the charged target molecules can move in lateral or diagonal directions towards the opposite ends of the substrate. In FIG. 35b, the charged molecules can now move in two perpendicular directions in the microarray substrate surface. By switching the four electrodes on and off in a designed sequence, the target molecules can be driven to contact all probes on the substrate.

FIG. 36 illustrates an embodiment for generating movement of target molecules by localized heating and/or cooling. An increase of temperature in a localized position in the hybridization liquid can cause the liquid at and near this location to expand and rise. In a cooled environment, the liquid then cools, contracts and descends. A convection driven circulation can be established by utilizing this heating/cooling fluid dynamic. A hybridization apparatus can be fabricated based on this principle. As illustrated in FIG. 36a, a Peltier heat pump is provided on the cover slip. The heat pump heats one position of the liquid while simultaneously cooling another position to establish a convective circulation between the two positions. The temperature change caused by such heating and cooling may be kept small so that the temperature remains within the range at which hybridization or associations of target and probe occurs. In other embodiments, the temperature differential need not be provided by a Peltier heat pump, and can be provided with any heating element and cooling element.

The establishment of temperature differential caused by the heat pump utilizes gravity to cause convective circulation. When the liquid layer between the cover slip and the substrate is very thin, vertical flow between cover slip and substrate may be difficult to establish. A way to establish more efficient convection is to stand the cover slip and substrate assembly on its edge during hybridization, as shown in FIG. 36b. Furthermore, a number of such heating-cooling pairs can be arranged across the microarray to establish multiple circulations. The heating-cooling poles can be reversed. In addition, phases of the heat-cool cycles among different pairs can be programmed to establish a “global” liquid circulation throughout the entire hybridization chamber.

For example, if the positions or temperatures of all heating-cooling pairs remain stable, a particular target molecule will be trapped in a local circulation around a particular pair. However, if, in the middle of a circulation, a temperature or position change is introduced to the pairs, a new circulation pattern will be established. By controlling the changing temperature or position, this method can be used to transport the molecule into a different circulation around a different pair. By alternating circulation patterns in a programmed fashion, any target molecule in the fluid can be transported anywhere on the substrate.

An effective interaction between the sample solution and the probes on the substrate can also be achieved by pneumatically driving the sample solution in and out of the hybridization chamber through microfluidic channels. In accordance with embodiments of this method, microfluidic channels are fabricated on a cover slip, which is placed on top of the microarray for hybridization with the micro-channels facing the array.

In one embodiment as illustrated in FIG. 37, probes on the microarray substrate may be arranged in such a way that there is extra space between columns or rows for wall portions found in the cover slide to contact the substrate to form channels without contacting probes. Open-top microfluidic channels are fabricated on the cover slip. The channels are patterned in such a way that when the cover slip is positioned over the microarray substrate, each probe column or row falls into a particular fluidic channel. A seal between the cover slip and the substrate slide can be formed by providing a thin gasket layer between the cover slide and the substrate slide. In this way, sample solutions can be pumped into and guided by the channels to interact with each probe along the channels.

The fluidic channels can be fabricated in the cover slip, for example, by etching a flat substrate or by a direct molding process. Many different channel designs are possible. FIGS. 37 and 38 illustrate two specific channel designs. Channels are linked to a reservoir at each end, either directly or through other channels. Each reservoir is exposed to a pressure chamber. By generating a pressure difference between the two pressure chambers, the sample liquid is driven back and forth through the channels, as illustrated in FIG. 39. A thorough interaction between the sample and probe can be achieved in an orderly fashion. Pressure can be generated in the pressure chamber by either pumping a gas or immiscible liquid in and out of the chambers. Alternatively a voltage can be applied between the two reservoirs that drives the target molecules back and forth through the microfluidic channels by electrophoresis mechanism.

As illustrated in FIG. 40, the micro-channels may in one embodiment of the invention form periodical spatial patterns across the entire microarray. The pitch of the micro-channel pattern can be equal to or much smaller than the size of a spot on the microarray. Assuming that the diameter of a probe spot on the microarray is D and the pitch of the microarray is P, the pitch of the micro-channels, p, can be P or D; or preferably 0.5D; or preferably, 0.2D; or more preferably 0.1D; or 0.05D; or 0.01D. The depth of the micro-channel, h, can be anything ranging from 10D to 0.0001D. The width of the micro-channel, w, can range from 99% to 1% of the pitch. When the micro-channel pitch is close to D, the width of the channel should take more than 90% of the pitch to ensure that most areas of a spot is covered by a channel. In a particular embodiment, the surfaces in the trenches of the microarray are made highly hydrophilic while the top of the “ridge” surface between two adjacent micro-channels is made hydrophobic (FIG. 41).

The micro-channels can have different spatial patterns across the surface of the cover slip. FIG. 42 shows a number of different designs. In FIG. 42a, the micro-channels are connected into a single channel zig-zag across the surface. In FIG. 42b, an array of parallel micro-channels are provided across the surface of the microarray. In FIG. 42c, the micro-channels are cross-connected to form a two-dimensional matrix of micro-channels. FIG. 42d shows another configuration of the two-dimensional cross-connected micro-channels, where the “ridges” are positioned to provide random or semi-random distribution of flow. Ridges in this configuration can be bumps, which can have different three-dimensional shapes, such as columns, diamonds, hemispheres, etc. Ridges in this configuration can be high enough that they are in contact with the microarray surface when the cover slip is placed on the microarray with the ridges facing the microarray surface. Alternatively, ridges can be lower so that they are not in contact with the microarray surface when the cover slip is placed on the microarray with the ridges facing the microarray surface. In this situation, ridges can help create turbulent flow of the liquid, and hybridization sensitivity and efficiency can be improved.

The cover slip can be made of any suitable material including, for example, glass, silicon, polymer, ceramic and metal. The micro-channels can be made of the same material as the cover slip or they can be made of a different material that is laminated or deposited on the cover slip substrate. The material forming the micro-channel can be hard or relatively soft (for example, polydimethyl siloxane (PDMS)). The micro-channel structure can be fabricated using, for example, one of the following micro-fabrication methods: etching (dry or wet), hot embossing, injection molding, micro-electronic discharge machining (EDM) or soft lithography. For example, the micro-channels can be fabricated in the cover slip by etching a flat substrate using precision etching as is found in semiconductor manufacturing. Alternatively, the micro-channels can be fabricated by pressing a patterned plate on the surface of the cover slip material at a temperature high enough to emboss the pattern of the plate onto the cover slip surface. The micro-channels can also be fabricated by injecting the molten substrate material and cooling the material in the mold.

In one embodiment of the invention, a clamping force can be exerted to the microarray substrate and the cover slip to ensure that the “ridges” of the micro-channel field are in firm contact with the microarray surface, as illustrated in FIG. 40. The sample liquid can be introduced into the channels before or after the placement of cover slip onto the microarray and it is pumped back and forth through the micro-channels during the hybridization.

To facilitate liquid pumping, reservoirs in fluid communication with the micro-channels can be formed on the cover slip. Liquid flow through the micro-channels can be generated by applying a positive or negative pressure to these reservoirs. There can be, for example, two reservoirs at each end of the cover slip, as shown in FIGS. 42-43. In other embodiments, more than two reservoirs are possible.

FIG. 43 shows one embodiment in which two reservoirs are provided at two ends of the cover slip. Each of these reservoirs includes a through hole connecting the reservoir to the surface of the cover slip opposite the hybridization chamber. On one end, a capillary is inserted into the hole and secured in place. The interior of the capillary therefore becomes part of the reservoir and can receive sample liquid that has passed through the micro-channels. At the opposite end, the other reservoir is coupled with a pressure control source, which provides a positive or negative pressure on that reservoir to cause the sample liquid to flow through the micro-channel. The position of the liquid-air interface in the capillary can be used to measure the volume of liquid that has been pumped through the micro-channels. The measurement can be used to maintain consistency between hybridizations and allow for repeatable hybridization processes.

In some cases, the area on the probe spot that is under the “ridge” part of the micro-channel may not produce any signal because it does not contact the sample liquid. However, in most microarray applications, the probe molecules are in vast over supply in comparison to sample molecules. Therefore, the portion of the probe spot covered by the “ridge” portion does not have a detrimental effect on the ability to detect hybridization in the microarray. However, in some instances it may be desirable to ensure that the total area available for hybridization is within a suitable coefficient of variation (CV) from spot to spot. A suitable CV can be less than 1%, less than 5%, less than 10%, less than 15%, less than 20%, or less than 25%. This can be achieved by either making the pitch of the micro-array much smaller than the size of the spot or making sure the width of ridge is much smaller than the pitch or the size of the spot. No precision alignment between the micro-channels on the cover slip and the microarray is necessary.

FIG. 44 shows the spot images with two different micro-channel configurations. In FIG. 44a, the channel pitch is similar to the diameter of the probe spot and the channel width is 90% of the pitch. Because the majority of the area on the spot is available for hybridization, the effect of the micro-channel structure on spot to spot uniformity is insignificant. In FIG. 44b, the channel pitch is much smaller than the diameter of the probe spot and the channel width is 50% of the pitch. Although the area available for hybridization is reduced by 50%, the spot to spot signal uniformity is not affected significantly because the channel pitch is much smaller than the spot size. As mentioned before, because the probe molecules are normally in vast oversupply in most applications, the reduction in hybridization area will not significantly affect the ability to detect hybridization in the microarray.

In a specific example, the diameter of the probe spot on the microarray is 100 μm, the micro-channels have a pitch of 10 μm and a depth and width of 1 μm and 7 μm, respectively. The total volume of liquid needed to fill the micro-channels across the entire cover slip is 0.98 μl. The total sample volume required for hybridization is smaller than 3 μl, even taking liquid pumping into consideration, which is much smaller than that required in most hybridization systems today (˜100 μl). The hybridization rate, hence the detection sensitivity can be greatly enhanced due to the increased sample concentration. In addition, because of the very small channel depth which greatly reduces the diffusion distance of target molecules in Z direction, the speed of the hybridization can also be greatly enhanced.

The micro-channel system described can be used for any liquid to liquid mixing. For example, a different liquid can be loaded into the reservoirs of a microarray and pumped into the micro-channels. By pumping back and forth through the micro-channels, different liquids can be mixed within the micro-channels. The system described can also be used to enhance interactions between target molecules in the liquid and molecules printed on or attached to the surface of the microarray.

FIG. 45 illustrates a method of generating movement of target molecules by applying pressure onto a flexible cover slip. In the embodiment shown, the cover slip is formed of an elastic material and one or more movable pins are positioned on top of the flexible cover slip. A tap on the cover slip by one of the pins generates a pressure wave in the liquid sample contained between the cover slip and the substrate slide. The motion of the pins can be programmed in such a way that the sample liquid is pumped to flow in a designed pattern, thus forcing the interaction between the target molecules and the probes. Flow patterns can be switched many times during hybridization to ensure thorough interaction. Pins may move in a vertical or lateral direction in the sample solution, or move in combinations of these two directions.

Alternatively, the hybridization can be performed without the cover slip, as illustrated in FIG. 46. A vibrating pin can be inserted into the liquid sample to improve hybridization directly. Pins can be coated with an inert material such as polytetrafluoroethylene (or Teflon®) to prevent the liquid sample from sticking to the pins. When the cover slip is not used, the hybridization process can be performed in a high humidity chamber to minimize evaporation.

C. Hybridization Apparatus Having an Inlet for Target Liquid Introduction

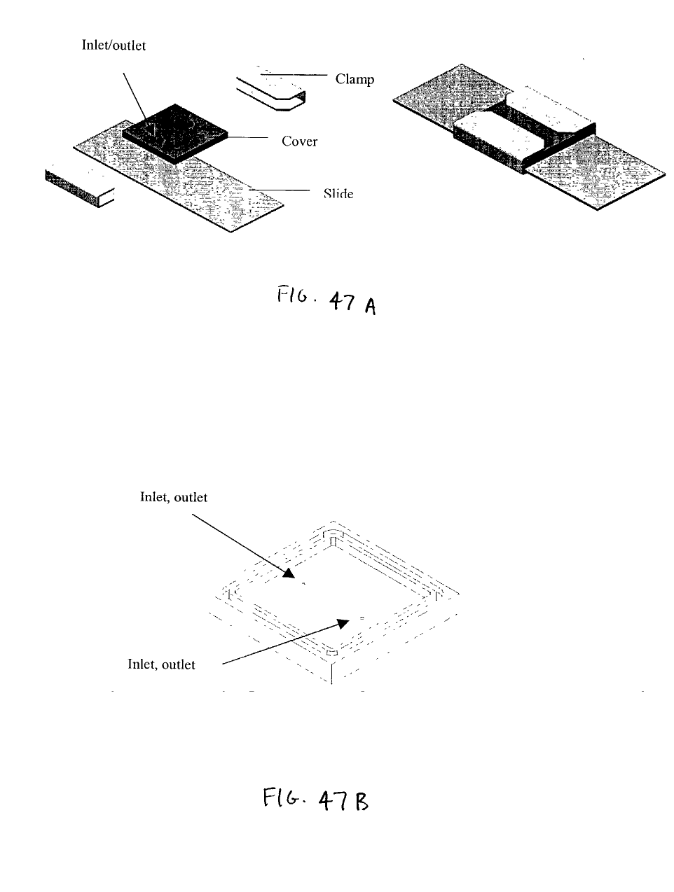

Embodiments of the present invention provide a hybridization apparatus including a hybridization chamber which creates turbulent flow of target liquid while shaking the apparatus so that effective movement of target molecules occurs during hybridization. The hybridization apparatus includes a substrate slide and a cover. The substrate slide has an array of probes deposited on its surface. The cover, as illustrated in one embodiment of this invention in FIG. 47, forms a hybridization chamber when it is placed on top of the substrate. The cover may have an adhesive bottom portion that can be firmly adhered on to the substrate slide surface covering the array, as shown in FIG. 48. Alternatively, the cover and the substrate slide may be clamped together by two clamps with a gasket on the bottom of the cover as shown in FIG. 47A. This hybridization apparatus can he shaken vigorously to generate turbulent flow in the target liquid.

The hybridization chamber may have, for example, inner chamber dimensions of 20 mm×20 mm×(1.0 mm through 1.75 mm) that take a sample volume of 350-500 μl with a void occupying the rest 100-200 μl equivalent volume in the chamber. This void can help to generate the turbulent flow in the chamber and thus improve hybridization rate and sensitivity.

In various embodiments, the material of the cover has the following characteristics: first, the material is substantially rigid so that the cover is not deformed in the presence of liquids; second, the material does not absorb target molecules in the sample such as DNA or fluorescent dyes; and third, the material is compatible with the chemicals in the hybridization mix. Materials such as polyethylene may be suitable for the cover. The gasket used may also possess the characteristics listed above. The inner surface of the cover can be coated with a hydrophobic material.

The cover may be provided with an opening as an inlet on one side or the top of the cover for introducing target liquid. The cover may have another opening as an outlet for removing the target liquid. The inlet and outlet can be closed, for example, by a clamp valve or rubber plugs. One can open the clamp valve or rubber plugs valve to introduce or remove the target liquid. The volume of the target liquid to be introduced should be slightly less than the volume of the chamber volume so that there is a small void in the chamber for allowing the formation of a turbulent flow and an effective movement of target liquid during shaking. The hybridization chamber can be shaken vigorously in a hybridization oven to create good turbulent flow.

Various embodiments of the present invention can reduce the hybridization set-up time. Since the substrate slide with the array of probes can be shipped with the cover affixed onto the slide, a user can simply add the prepared target liquid directly into the chamber and hybridize the target to the probes in an oven with a shaker. After the hybridization process, the cover can be removed from the substrate slide. The substrate slide can then be washed and read. This can significantly reduce the delay in proceeding to the next step after the hybridization.

D. Antibacterial Screening Having Improved Fluid Interaction

In accordance with embodiments of the present invention, antibacterial screening systems and methods having improved fluid interaction and mixing are provided. In one example, an array of suspected antimicrobial compounds are deposited onto a substrate slide as described in the various embodiments above. The targeted bacterial microbes in solution are deposited onto the array of suspected antimicrobial compounds on the substrate slide either before or after the substrate slide is mated with a corresponding cover slip. Next, any of the above-described systems and methods can be used to cause the microbe solution to flow, thereby facilitating the effective mixing of the targeted bacteria microbe solution with the array of suspected antimicrobial compounds.

Finally, after the microbe solution has thoroughly mixed with the suspected antimicrobial compounds, the cover slip can be removed to permit examination to determine whether any zones of inhibition have formed on each of the compounds in the microarray. Optical inspection can be used to determine the existence and extent of antibacterial activity.

In alternative embodiments, the systems and methods described above with respect to antibacterial assays can also be applied to antifungal assays.

The various apparatus and methods described above can be applicable for the detection of any specific interactions between biological or chemical molecules, including associations, hybridizations, and reactions between molecules. Examples of such associations that can be investigated by embodiments of this invention include, but are not restricted to, complementary DNA-DNA association, complementary DNA-RNA association, protein-protein association, peptide-protein association, antigen-antibody association, ligand-receptor association, agonist- or antagonist-receptor association, substrate- or cofactor-enzyme association and reaction.

All publications and patent applications cited in this specification are incorporated by reference herein in their entirety as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A microarray apparatus, comprising:
a substrate having an array of probes deposited on a surface of the substrate for interaction with a target molecule in a target liquid; and
a cover coupled to the substrate to form a reaction chamber therebetween, wherein the array of probes is contained within the reaction chamber and the substrate and the cover are movable relative to each other.

2. The microarray apparatus of claim 1, wherein the substrate is fixed and the cover is movable.

3. The microarray apparatus of claim 1, wherein the cover is fixed and the substrate is movable.

4. The microarray apparatus of claim 1, wherein both the substrate and cover are movable.

5. The microarray apparatus of claim 1, further comprising:
a first liquid confinement coating on the substrate for retaining the target liquid in a first predetermined region encompassing the array of probes.

6. The microarray apparatus of claim 5, wherein the cover has a hydrophobic coating.

7. The microarray apparatus of claim 5, wherein:
said first liquid confinement coating comprises a first hydrophilic region containing the array of probes and a first hydrophobic region surrounding the first hydrophilic region.

8. The microarray apparatus of claim 7, further comprising:
a second liquid confinement coating on the cover, said second liquid confinement coating comprising a second hydrophilic region aligned with the first hydrophilic region on the substrate and a second hydrophobic region aligned with the first hydrophobic region on the substrate.

9. The microarray apparatus of claim 1, further comprising:
a substrate holder for retaining the substrate;
a cover holder for retaining the cover; and
an agitator for agitating the substrate holder and the cover holder to induce relative movement between the substrate and the cover.

10. The microarray apparatus of claim 9, wherein:
said substrate holder comprises one or more barriers for preventing movement of the substrate relative to the substrate holder when the agitator is agitating the substrate holder and the cover holder; and
said cover holder comprises one or more barriers for allowing limited movement of the cover relative to the cover holder when the agitator is agitating the substrate holder and the cover holder.

11. The microarray apparatus of claim 9, further comprising:
said substrate holder comprises one or more barriers for allowing limited movement of the substrate relative to the substrate holder when the agitator is agitating the substrate holder and the cover holder; and
said cover holder comprises one or more barriers for preventing movement of the cover relative to the cover holder when the agitator is agitating the substrate holder and the cover holder.

12. The microarray apparatus of claim 1, further comprising:
a liquid confinement coating on the cover, said liquid confinement coating comprising a hydrophilic region aligned with the array of probes on the substrate and a hydrophobic region surrounding the hydrophilic region.

13. The microarray apparatus of claim 1, wherein:
said cover further comprises protrusions extending into the reaction chamber for agitating the target liquid when the cover and the substrate are moved relative to each other.

14. The microarray apparatus of claim 13, wherein:
each of said protrusions is shaped to preferably induce flow in one direction as the cover is agitated.

15. The microarray apparatus of claim 14, wherein:
each of said protrusions comprises a shaped ridge.

16. The microarray apparatus of claim 1, further comprising:
an agitator for moving the cover relative to the substrate.

17. The microarray apparatus of claim 16, wherein:
said agitator mechanically moves the cover relative to the substrate.

18. The microarray apparatus of claim 16, wherein:
said cover is magnetically reactive; and
said agitator generates a movable magnetic field for moving the cover.

19. The microarray apparatus of claim 18, wherein:
said movable magnetic field generated by the agitator moves the cover in a circular motion.

20. The microarray apparatus of claim 1, wherein the substrate is a carrier having the array of probes deposited on a surface of the carrier, and the cover has risers on a surface that form a container having a size slightly larger than the carrier so that when the carrier is placed in the container and a target liquid is placed in the container the array of probes deposited on the surface of the carrier is in contact with the target liquid, and wherein the carrier or the cover is attached to a motor so that a relative motion between the carrier and the cover can be introduced.

21. The microarray apparatus of claim 5, comprising:
a second array of probes deposited on the surface of the substrate;
wherein the first liquid confinement coating is further configured to retain a second quantity of target liquid in a second predetermined region encompassing the second array of probes and to prevent mixing of the target liquid retained in the first predetermined region with the second quantity of target liquid in the second predetermined region.

22. The microarray apparatus of claim 21, wherein:
said first liquid confinement coating comprises:
a first hydrophilic region containing the array of probes and a first hydrophobic region surrounding the first hydrophilic region; and
a second hydrophilic region containing the second array of probes and a second hydrophobic region surrounding the second hydrophilic region.

23. The microarray apparatus of claim 22, further comprising:
a second liquid confinement coating on the cover, said second liquid confinement coating comprising:
a third hydrophilic region aligned with the first hydrophilic region on the substrate;
a third hydrophobic region aligned with the first hydrophobic region on the substrate;
a fourth hydrophilic region aligned with the second hydrophilic region on the substrate; and
a fourth hydrophobic region aligned with the second hydrophobic region on the substrate.

24. The microarray apparatus of claim 1, wherein:
said array of probes comprises an array of suspected antimicrobial compounds; and
said target molecules comprise bacterial microbes.

25. A microarray apparatus, comprising:
a reaction chamber having an interior cavity and an array of probes deposited on an inner surface of the interior cavity for reaction with a target molecule in a target liquid; and

26. The microarray apparatus of claim 25, further comprising:
a magnetically reactive mixing member contained in the reaction chamber; and
a magnetic field generator for moving the magnetically reactive mixing member through the target liquid.

27. The microarray apparatus of claim 26, wherein said reaction chamber further comprises:
a substrate having the array of probes deposited thereon; and
a cover coupled to the substrate to form the interior cavity of the reaction chamber.

28. The microarray apparatus of claim 27, further comprising:
a sealing layer coupled between the substrate and the cover, said sealing layer defining an aperture such that the cover, the aperture in the sealing layer, and the substrate form the interior cavity of the reaction chamber.

29. The microarray apparatus of claim 26, wherein the magnetically reactive mixing member comprises one or more magnetic particles.

30. The microarray apparatus of claim 26, wherein the magnetically reactive mixing member comprises a magnetic volume exclusion liquid.

31. A microarray apparatus, comprising:
a reaction chamber having an interior cavity;
a target liquid contained within the interior cavity of the reaction chamber;
a volume exclusion liquid contained within the interior cavity; and
an array of probes deposited on an inner surface of the interior cavity of the reaction chamber for reaction with a target molecule in the target liquid.

32. The microarray apparatus of claim 31, wherein said target liquid has a different density than the volume exclusion liquid.

33. The microarray apparatus of claim 31, wherein said target liquid is substantially immiscible with the volume exclusion liquid.

34. The microarray apparatus of claim 31, wherein said target liquid is magnetic.

35. The microarray apparatus of claim 31, wherein said volume exclusion liquid is magnetic.

36. The microarray apparatus of claim 31, wherein said reaction chamber further comprises:
a substrate having the array of probes deposited thereon; and
a cover coupled to the substrate to form the interior cavity of the reaction chamber therebetween.

37. The microarray apparatus of claim 36, further comprising:
a sealing layer coupled between the substrate and the cover, said sealing layer defining an aperture such that the cover, the aperture in the sealing layer, and the substrate form the interior cavity of the reaction chamber.

38. The microarray apparatus of claim 31, further comprising an agitator for agitating the reaction chamber to cause the target liquid and the volume exclusion liquid to move relative to the array of probes.

39. The microarray apparatus of claim 38, wherein said agitator comprises a centrifuge.

40. A microarray apparatus, comprising:
a reaction chamber having an interior cavity;
an array of probes deposited on an inner surface of the interior cavity for reaction with a target molecule in a target liquid; and
a transducer for directing acoustic waves into the interior cavity of the reaction chamber.

41. The microarray apparatus of claim 40, wherein said transducer generates ultrasonic waves.

42. The microarray apparatus of claim 40, wherein said reaction chamber comprises:
a substrate having the array of probes deposited thereon; and
a cover coupled to the substrate to form the interior cavity of the reaction chamber therebetween.

43. The microarray apparatus of claim 42, further comprising:
a sealing layer coupled between the substrate and the cover, said sealing layer defining an aperture such that the cover, the aperture in the sealing layer, and the substrate form the interior cavity of the reaction chamber.

44. A microarray apparatus, comprising:
a reaction chamber having an interior cavity;
an array of probes deposited on an inner surface of the interior cavity for reaction with a charged target molecule in a target liquid; and
a voltage generator for generating a voltage across the interior cavity to move the charged target molecule.

45. The microarray apparatus of claim 44, wherein said voltage generator comprises a plurality of electrical leads positioned around the interior cavity of the reaction chamber.

46. The microarray apparatus of claim 45, wherein said plurality of electrical leads extend into the interior cavity of the reaction chamber.

47. The microarray apparatus of claim 44, wherein said reaction chamber further comprises:
a substrate having the array of probes deposited thereon; and
a cover coupled to the substrate to form the interior cavity of the reaction chamber therebetween.

48. The microarray apparatus of claim 47, further comprising:
a sealing layer coupled between the substrate and the cover, said sealing layer defining an aperture such that the cover, the aperture in the sealing layer, and the substrate form the interior cavity of the reaction chamber.

49. The microarray apparatus of claim 44, wherein:
said voltage generator is configured to reverse the voltage across the interior cavity according to a predetermined pattern.

50. The microarray apparatus of claim 44, further comprising:
a magnetic field generator for generating a magnetic field across the interior cavity of the reaction chamber in a first direction;
wherein said voltage generator is configured to generate an electric field across the interior cavity of the reaction chamber in a second direction, said second direction being non-parallel with the first direction.

51. A microarray apparatus, comprising:
a reaction chamber having an interior cavity;
an array of probes deposited on an inner surface of the interior cavity for reaction with a charged target molecule in a target liquid; and
a temperature control mechanism for generating a temperature gradient across the interior cavity of the reaction chamber.

52. The microarray apparatus of claim 51, wherein:
said temperature control mechanism comprises a heat pump for heating a first portion of the interior cavity and cooling a second portion of the interior cavity.

53. The microarray apparatus of claim 52, wherein:
said reaction chamber comprises:
a substrate having the array of probes deposited thereon; and
a cover coupled to the substrate to form the interior cavity of the reaction chamber therebetween; and
said heat pump comprises a heating element provided at a first location of the cover and a cooling element provided at a second location of the cover.

54. The microarray apparatus of claim 53, wherein:
said cover is oriented in a vertical direction with the heating element positioned below the cooling element such that target fluid heated by the heating element rises from the heating element to the cooling element, where the target fluid is cooled by the cooling element and is drawn down to the heating element by gravity.

55. A microarray apparatus, comprising:
a substrate;
an array of probes deposited on a surface of the substrate; and
a cover having a channel with a width smaller than a width of the array of probes, said cover being coupled to the substrate such that said channel and said substrate define a channel cavity such that a target fluid flowing through the channel cavity contacts each probe in the array of probes.

56. The microarray apparatus of claim 55, further comprising:
a flow inducer for inducing a target fluid to flow through the channel cavity across the array of probes.

57. The microarray apparatus of claim 56, wherein:
said channel has a first end and a second end; and
said flow inducer comprises a pressure generator for generating a pressure difference between the first and second ends of the channel such that the target liquid is driven back and forth through the channel cavity.

58. The microarray apparatus of claim 55, wherein each probe is completely contained within the channel cavity.

59. The microarray apparatus of claim 55, wherein each probe is partially contained within the channel cavity.

60. The microarray apparatus of claim 59, wherein a portion of each probe is contained within the channel cavity, wherein the portion of each probe that is contained within the channel cavity has coefficient of variation less than about 25% from probe to probe.

61. The microarray apparatus of claim 60, wherein the coefficient of variation is less than about 10%.

62. The microarray apparatus of claim 60, wherein the coefficient of variation is less than about 5%.

63. The microarray apparatus of claim 60, wherein the coefficient of variation is less than about 1%.

64. A microarray apparatus, comprising:
a reaction chamber having an interior cavity;
an array of probes deposited on an inner surface of the interior cavity of the reaction chamber for reaction with a target molecule in a target liquid; and
a shape modulator for varying the shape of the interior cavity.

65. The microarray apparatus of claim 64, wherein:
said shape modulator comprises one or more movable protrusions, each of said protrusions being extendible into the interior cavity of the reaction chamber.

66. The microarray apparatus of claim 44, wherein:
at least a portion of the reaction chamber is flexible; and
said shape modulator comprises one or more movable protrusions, each of said protrusions being extendible to deform the flexible portion of the reaction chamber.

67. A microarray apparatus comprising a chamber filled with a combination of a volume exclusion liquid and a target liquid.

68. The microarray apparatus of claim 67, wherein the volume exclusion liquid is a magnetic liquid.

69. A microarray apparatus comprising:
a substrate having a plurality of arrays of probes deposited on a surface the substrate; and
a cover coupled with the substrate such that the cover and the substrate form a chamber over each array of probes, said cover having an inlet for introducing a target liquid into the chamber.

70. The microarray apparatus of claim 69, further comprising a clamp for coupling the cover with the substrate.

71. The microarray apparatus of claim 69, wherein the cover has an outlet for removing the target liquid.

72. A method for promoting interaction between a target molecule in a target liquid and an array of probes deposited on a surface of a substrate, said method comprising:
loading the target liquid on top of the array of probes;
positioning a cover on top of the target liquid; and
creating a relative motion between the substrate and the cover for generating movement of the target molecule.

73. The method of claim 72, wherein said creating the relative motion between the substrate and cover comprises immobilizing the substrate and moving the cover.

74. The method of claim 72, wherein said creating the relative motion between the substrate and cover comprises immobilizing the cover and moving the substrate.

75. The method of claim 72, wherein said creating the relative motion between the substrate and cover comprises moving the substrate and the cover.

76. The method of claim 72, further comprising:
retaining the substrate in a substrate holder; and
retaining the cover in a cover holder.

77. The method of claim 76, wherein:
either said cover holder permits limited movement of the cover within the cover holder or said substrate holder permits limited movement of the substrate within the substrate holder; and
said creating the relative motion between the substrate and cover comprises agitating the cover holder and the substrate holder to cause relative movement between the cover and the substrate.

78. The method of claim 72, further comprising confining the target liquid within a confinement area around the array of probes.

79. The method of claim 72, wherein said confining the target liquid is accomplished by creating a surface tension differential on the surface of the substrate.

80. The method of claim 72, wherein said confining the target liquid is accomplished by creating a surface tension differential on the surface of the cover.

81. The method of claim 72, wherein:
said cover is magnetically reactive; and
said creating the relative motion between the substrate and the cover comprises applying a magnetic force to the cover.

82. The method of claim 72, wherein:
a plurality of arrays of probes are deposited on the substrate surface;
said loading the target liquid comprises loading a first portion of target liquid into a first confinement area around a first array of probes and loading a second portion of target liquid into a second confinement area around a second array of probes; and
said confining the target liquid within the confinement area comprises inhibiting the first portion of target liquid from mixing with the second portion of target liquid.

83. The method of claim 82, wherein:
said confinement area comprises a first hydrophilic coating surrounded by a first hydrophobic coating, the first array of probes being deposited on the first hydrophilic coating; and
said second confinement area comprises a second hydrophilic coating surrounded by a second hydrophobic coating, the second array of probes being deposited on the second hydrophilic coating.

84. The method of claim 82, wherein:
said loading the target liquid comprises loading the target liquid containing target bacterial microbes on top of an array of suspected antimicrobial compounds.

85. The method of claim 82, wherein the cover includes a third confinement area aligned with the first confinement area and a fourth confinement area aligned with the second confinement area.

86. A method for promoting interaction between a target molecule in a target liquid and an array of probes deposited on an interior surface of a reaction chamber for confining the target liquid, said method comprising:
loading the target liquid in the reaction chamber; and
applying a magnetic force to move a magnetically reactive mixing member contained within the reaction chamber to generate motion of the target molecule.

87. The method of claim 86, wherein:
said magnetically reactive mixing member comprises one or more magnetically reactive particles.

88. The method of claim 86, wherein:
said magnetically reactive mixing member comprises a magnetically reactive volume exclusion liquid.

89. A method for promoting interaction between a target molecule in a target liquid and an array of probes deposited on an interior surface of a reaction chamber for confining the target liquid, said method comprising:
loading the target liquid into the reaction chamber;
loading a volume exclusion liquid into the reaction chamber; and
agitating the reaction chamber to cause relative movement between the volume exclusion liquid and the target liquid.

90. The method of claim 89, wherein said agitating the reaction chamber comprises rotating the reaction chamber.

91. The method of claim 89, further comprising applying a centrifugal force to the reaction chamber while rotating the reaction chamber.

92. A method for promoting interaction between a target molecule in a target liquid and an array of probes deposited on an interior surface of a reaction chamber, said method comprising:
loading the target liquid into the reaction chamber; and
directing acoustic waves through the target liquid to generate motion of the target molecule.

93. A method for promoting interaction between a charged target molecule in a target liquid and an array of probes deposited on a surface of a substrate, said method comprising:
loading the target liquid into the reaction chamber; and
generating an electric field across the reaction chamber to generate motion of the charged target molecule contained within the target liquid.

94. The method of claim 93, further comprising:
modulating the electric field across the reaction chamber to move the charged target molecule in a desired pattern.

95. A method for promoting interaction between a target molecule in a target liquid and an array of probes deposited on an interior surface of a reaction chamber for confining the target liquid, said method comprising:
loading the target liquid in the reaction chamber; and
generating a temperature gradient in the target fluid across the reaction chamber.

96. The method of claim 95, wherein said generating the temperature gradient comprises:
heating a first portion of the reaction chamber; and
cooling to a second portion of the reaction chamber.

97. The method of claim 96, further comprising:
positioning the heated first portion of the reaction chamber below the cooled second portion of the reaction chamber such that target fluid heated in the first portion of the reaction chamber rises from the first portion to the second portion, where the target fluid is cooled and drawn back to the first portion by gravity.

98. A method for promoting interaction between a target molecule in a target liquid and an array of probes deposited on a surface of a substrate, said method comprising:
loading a target liquid into a channel, said channel having a width smaller than a width of the array of probes;
passing the target liquid through the channel across all of the probes in the probe array.

99. The method of claim 98, wherein:
said passing the target liquid through the channel comprises generating a pressure differential between a first end of the channel and a second end of the channel.

100. The method of claim 99, wherein:
said passing the target liquid through the channel further comprises reversing the pressure differential between the first end of the channel and the second end of the channel.

101. The method of claim 98, wherein said passing the target liquid through the channel comprises passing the target liquid across the entirety of each probe in the probe array.

102. The method of claim 98, wherein said passing the target liquid through the channel comprises passing the target liquid across a portion of each probe in the probe array.

103. The method of claim 102, wherein the portion of each probe across which the target liquid passes has coefficient of variation less than about 25% from probe to probe.

104. The method of claim 103, wherein the coefficient of variation is less than about 10%.

105. The method of claim 103, wherein the coefficient of variation is less than about 5%.

106. The method of claim 103, wherein the coefficient of variation is less than about 1%.

107. A method for promoting interaction between a target molecule in a target liquid and an array of probes deposited on an interior surface of a reaction chamber for confining the target liquid, said method comprising:
loading the target liquid into an interior cavity of the reaction chamber; and
changing the shape of the interior cavity of the reaction chamber to generate a pressure wave in the target liquid.

108. The method of claim 107, wherein said changing the shape of the interior cavity comprises extending protrusions into the interior cavity.

109. The method of claim 107, wherein said changing the shape of the interior cavity comprises applying a force to a flexible member forming at least a portion of the reaction chamber.

110. The method of claim 107, wherein said loading the target liquid into the interior cavity of the reaction chamber comprises:
loading the target liquid onto an array of probes deposited on a surface of a substrate slide; and
coupling the substrate slide with a cover, at least a portion of the cover formed of a flexible member.

111. A microarray apparatus, comprising:
a reaction chamber comprising a substrate having an array of probes deposited thereon, and a cover coupled to the substrate to form an interior cavity of the reaction chamber between the substrate and the cover;
an array of probes deposited on an inner surface of the interior cavity for reaction with a charged target molecule in a target liquid; and
a flow inducing mechanism for inducing flow of the target liquid without physically translating either the substrate or the cover.

112. The microarray apparatus of claim 111, wherein:
said flow inducing mechanism comprises a transducer for directing acoustic waves into the interior cavity of the reaction chamber.

113. The microarray apparatus of claim 111, wherein:
said target molecule is charged; and
said flow inducing mechanism comprises an electric field generator for generating an electric field across the interior cavity to move the charged target molecule.

114. The microarray apparatus of claim 111, wherein:
said flow inducing mechanism comprises a temperature control mechanism for generating a temperature gradient across the interior cavity of the reaction chamber.

115. A method for promoting interaction between an array of probes deposited on a surface of a substrate and a target molecule in a target liquid contained within a reaction chamber formed by the substrate and a cover, said method comprising:
loading the target liquid in the reaction chamber; and
inducing movement of the target molecules in the target liquid without physically translating either the substrate or the cover.

116. The method of claim 115, wherein:
said inducing movement comprises directing acoustic waves into the reaction chamber.

117. The method of claim 115, wherein:
said target molecule is charged; and
said inducing movement comprises generating an electric field across the reaction chamber to move the charged target molecule.

118. The method of claim 115, wherein:
said inducing movement comprises generating a temperature gradient across the reaction chamber.
